(19) United States
(12) Patent Application Publication
(10) Pub. No.: US 2024/0347200 A1
(43) Pub. Date: Oct. 17, 2024

(54) SYSTEMS AND METHODS FOR EARLY-STAGE CANCER DETECTION AND SUBTYPING

(71) Applicant: Exai Bio, Inc., Palo Alto, CA (US)

(72) Inventors: Babak Alipanahi, Palo Alto, CA (US); Hani Goodarzi, Palo Alto, CA (US); Fereydoun Hormozdiari, Palo Alto, CA (US); Mehran Karimzadeh, Palo Alto, CA (US)

(21) Appl. No.: 18/636,128

(22) Filed: Apr. 15, 2024

Related U.S. Application Data
(60) Provisional application No. 63/496,344, filed on Apr. 14, 2023.

(51) Int. Cl. G16H 50/20; G06N 3/0455; G06N 3/0895; G16B 40/20

(52) U.S. Cl. CPC G16H 50/20; G06N 3/0455; G06N 3/0895; G16B 40/20

(57) ABSTRACT

Embodiments described herein provide a neural network based cancer detection and subtyping tool for predicting the presence of a tumor, its tissue of origin, and its subtype using small RNA sequencing (smRNA-seq) data, for example, the oncRNA count data. Specifically, the AI-based cancer detection and subtyping tool uses variational Bayes inference and semi-supervised training to adjust for batch effects and learn a low dimensional distribution explaining biological variability of the data. A method is also provided for determining the likely subtype(s) in a cancer sample.

### CROSS REFERENCE(S)

The instant application is a nonprovisional of and claims priority under 35 U.S.C. 119 to U.S. provisional application No. 63/496,344, filed Apr. 14, 2023, which is hereby expressly incorporated by reference in its entirety.

### TECHNICAL FIELD

The embodiments relate generally to artificial intelligence (AI) based diagnostics, and more specifically to systems and methods for AI-based early-stage cancer detection and subtyping using orphan non-coding ribonucleic acid (oncRNA) count data in biological samples of a subject.

### BACKGROUND

Recent medical research has shown that oncRNAs, a category of small RNAs (smRNAs) that are present in tumors and largely absent in healthy tissue, can be used in early stage cancer diagnostics. For example, the detection of the presence, absence, and/or quantity of oncRNAs or functional fragment thereof in a sample of a patient can be used in diagnosing and subtyping cancer for the patient. Some existing statistical methods such as ensemble logistic regression models, penalized logistic regression models and linear regression models have been trained to predict a diagnostic output based on an available oncRNA count input. However, linear models are often inefficient or even deficient in adjusting for batch effects, e.g., data variations caused by technical and non-biological factors from data sources, or modeling dependencies and interactions among the independent variables.

### BRIEF DESCRIPTION OF THE DRAWINGS

FIG. 1 is a simplified diagram illustrating a process of using an artificial intelligence (AI)-based diagnostic platform for cancer detection and subtyping, according to some embodiments described herein.

FIG. 2 is a simplified diagram illustrating an example architecture of the neural network model described in FIG. 1, according to embodiments described herein.

FIGS. 3A-3B provide simplified block diagrams illustrating example aspects of a two-phase training process of the AI-based cancer detection and subtyping tool, according to embodiments described herein.

FIG. 4 provides a simplified block diagram illustrating representative aspects of the inference/testing process of the AI-based cancer detection and subtyping tool, according to embodiments described herein.

FIG. 5 provides a simplified block diagram illustrating the data augmentation of RNA data from a training dataset, according to embodiments described herein.

FIG. 6 is a simplified diagram illustrating a computing device implementing an AI-based cancer detection and subtyping module, according to one embodiment described herein.

FIG. 7 is a simplified diagram illustrating the neural network structure implementing the cancer detection and subtyping module described in FIG. 6, according to some embodiments.

FIG. 8 is a simplified block diagram of a networked system suitable for implementing the cancer detection and subtyping framework described in FIGS. 1-5 and other embodiments described herein.

FIGS. 9A-9B provide an example logic flow diagram illustrating a method of training a neural network based model for generating a cancer diagnostic prediction based on the framework shown in FIGS. 1-5, according to some embodiments described herein.

FIG. 10 provides an example logic flow diagram illustrating a method of subtyping a lung cancer sample via a neural network based model based on the framework shown in FIGS. 1-5, according to some embodiments described herein.

FIGS. 11-14B provide example data plots illustrating example performance of the framework shown in FIGS. 1-5, according to some embodiments described herein.

Embodiments of the disclosure and their advantages are best understood by referring to the detailed description that follows. It should be appreciated that like reference numerals are used to identify like elements illustrated in one or more of the figures, wherein showings therein are for purposes of illustrating embodiments of the disclosure and not for purposes of limiting the same.

### DETAILED DESCRIPTION

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. In some embodiments, the module may be implemented on one or more neural networks.

As used herein, the term “small RNA” refers to an RNA species that is generally less than about 200 nt, for example in the range of 50-100 nt.

As used herein, the term “oncRNA” refers to a category of small RNAs (smRNAs), typically small non-coding RNAs (small ncRNAs) that are present in tumors but largely absent in healthy tissue. By way of non-limiting example only, oncRNAs may refer to small ncRNAs that (i) have a CPM below 0.09 in 95% of normal serum samples; (ii) have an adjusted p-value of <0.1 following an association study of tumor tissue versus normal tissues using a generalized linear model and correcting for known confounders, including age and sex; and (iii) as for small RNAs generally, are less than about 200 nt in length, such as in the range of 50-100 nt. In addition, while an oncRNA species is a non-coding RNA sequence, it may overlap, in part, with an adjacent coding sequence. Representative embodiments of detection and/or quantification of oncRNA molecules in a sample of a subject may be found in PCT International Application Pub. WO 2022/040106, which is hereby expressly incorporated by reference herein in its entirety.

Small RNAs can be secreted in cell-derived extracellular vesicles such as exosomes. Both mRNA and small non-coding RNA species have been found in extracellular vesicles. As such, extracellular vesicles can provide a vehicle for transfer and protection of RNA content from degradation in the extracellular environment, enabling a stable source for reliable detection of RNA biomarkers. Small ncRNA species can serve as “oncRNA” biomarkers when they are found to be differentially present in biological samples derived from subjects having cancer, as compared with subjects who are “normal,” i.e., subjects who do not have cancer. A small ncRNA species, or a set of small ncRNA species, is differentially present between samples if the difference between the levels of expression in cancer cells and normal cells is determined to be statistically significant. Common tests for statistical significance include, but are not limited to, t-test, ANOVA, Kruskal-Wallis, Wilcoxon, Mann-Whitney, Chi-squared, and Fisher's exact test. OncRNA biomarkers, alone or in combination, can be used to provide a measure of the relative likelihood that a subject has or does not have cancer.

In one implementation, small ncRNA biomarkers of cancer, i.e., oncRNAs, may be discovered by total and/or small RNA sequencing of multiple cancer types and subtypes from various tissues of origin, and identifying known or previously unknown small ncRNAs that are specifically expressed in cancer cells. Over 260,000 such RNAs have been identified across various cell/tissue and corresponding cancer types. These oncRNA biomarkers can be used to determine the type of cancer and cancer status of a subject, for example, a subject whose cancer status was previously unknown or who is suspected to be suffering from cancer. This may be accomplished by determining the level of one or more oncRNAs, or combinations thereof, in a biological sample derived from the subject. A difference in the level of one or more of these oncRNA biomarkers as compared to that in a biological sample derived from a normal subject is an indication that the subject has cancer of the type and tissue of origin associated with the oncRNA biomarkers. The method may also be carried out by determining the presence or absence of one or more of the identified oncRNAs as well as the absolute number of detected RNA species, i.e., RNA species that are distinct (different) from each other, wherein the absolute number of detected RNA species may be the absolute number of total RNA species, total small RNA species (i.e., below some specified maximum length), and/or total small ncRNA species. Any two or more of these methods can be used to analyze the same biological sample; that is, the sample may be analyzed with respect to (1) the levels of particular oncRNA biomarkers, (2) the presence or absence of such biomarkers, and/or (3) the absolute number of detected RNA species in the sample.

Existing statistical methods such as ensemble logistic regression models, penalized logistic regression models and linear regression models have been trained to predict a diagnostic output based on RNA count input, generally comprising at least an oncRNA count input. However, linear models are often inefficient or even deficient in adjusting for batch effects, e.g., data variations caused by technical and non-biological factors from data sources, or modeling dependencies and interactions among the independent variables. These drawbacks of linear models may manifest significantly when these linear models are trained on a dataset comprising multiple smaller datasets of RNA cancer research data from different data sources, which often cause batch effects due to the different suppliers, data sources, and other sources of variation.

In addition, as usually only a fraction of oncRNAs may be present in the volume of a blood draw, small RNA (smRNA) fingerprinting results in sparse patterns from thousands of individual oncRNAs species. Given the zero-inflated nature of oncRNA patterns, the underlying biological variation distinguishing different cancer types or separating cancer from non-cancer may become dominated by technical confounders, such as differences in sequencing depth, RNA extraction, sample processing, and other unknown sources of variation. In addition, often the sample collection process itself involves known sources of variation that should be accounted for, including biological differences between donors (age, sex, BMI, etc.). Therefore, developing a generalizable liquid biopsy assay may need to account for the biological properties of circulating biomarkers of interest and disentangling the technical and biological variation in sequencing data.

In view of the challenges in developing a robust oncRNA-based diagnostic tool, embodiments described herein provide a neural network based cancer detection and subtyping tool for predicting the presence of a tumor, its tissue of origin, and/or its subtype using RNA sequencing (smRNA-seq) data, for example, the oncRNA count data and the total small RNA count data. Specifically, the AI-based cancer detection and subtyping tool is built on a variational autoencoder (VAE) that encodes input RNA count data into a latent variable and one or more decoder heads (e.g., classification heads) to generate a prediction output such as the presence of the tumor, tissue of origin, subtype classification, and/or the like.

In addition to its utility in cancer detection, subtyping, and tissue identification, the present method is useful in at least the following: detecting cancer stage (e.g., TNM stage or number stage); detecting cancer pathway alterations (e.g., alterations in mitogenic signaling pathways, metabolic pathways, or DNA repair); detecting genomic aberrations; analyzing cancer cell state; detecting and analyzing aspects of various oncogenic processes (e.g., germline pathogenic variants, copy number alterations, and mutations such as somatic driver mutations); and detecting and analyzing other factors relevant to the detection or characterization of cancers.

The input RNA count data may be total RNA count and/or the count of small RNAs, miRNAs, mRNAs, small ncRNAs, small ncRNAs previously identified as oncRNAs, or the like. The term “input RNA count” is used herein to refer to any or all of the foregoing. For instance, in one embodiment, the input RNA count data comprises oncRNA count and (total) small ncRNA count. In another representative example, the input RNA count data comprises endogenous highly expressed RNA biotype count and oncRNA count. In an additional representative example, the input RNA count data includes oncKmer data, wherein an “oncKmer” or “onc K-mer” refers to an RNA sequence of size k that is significantly enriched in sequencing reads of small RNA sequencing (smRNA-seq) reads of tumor versus normal samples. The term “onc k-mer,” as used herein, generally refers to a k-mer of size k that is enriched (by a statistically significant difference) in small RNA sequencing (smRNA-seq) reads of cancer-derived samples as compared to non-cancer-derived samples. The length of k-mers may vary, for example, about 5, 10, 11, 12, 13, 14, 15, 20, 25, 30, 35, 40, 45, 50, 100, or 200 nucleotides in length, where the foregoing number of nucleotides in the k-mer can be exact nucleotide values or approximate nucleotide values.

In one embodiment, the prediction output may take a form of a probability distribution, e.g., P (presence of tumor=Yes|oncRNA count) and P (presence of tumor =No|oncRNA count), and/or the like. An arg max operation may be performed at the probability distribution to generate the final classification output. As another example, for the prediction of the presence of the tumor, a threshold may be applied, e.g., when P (presence of tumor=Yes|oncRNA count)>Th, a presence of cancer is determined. The prediction outputs for the tissue of origin or subtype may be obtained in a similar manner.

As another example, given an input oncRNA count, the AI-based cancer detection and subtyping tool may generate a prediction of cancer subtype, thus differentiating between two or more subtypes of a particular type of cancer, each of which displays different pathological and/or genetic signatures. By way of example and not limitation, the present method may be used to identify:

Breast cancer subtypes, including estrogen receptor positive (ER+), progesterone receptor positive (PR+), human epidermal growth factor receptor positive (HER2+), and triple-negative (TNBC), the latter characterized by the lack of expression of any of the aforementioned hormone receptors;

Colorectal cancer (CRC) subtypes, including those exhibiting chromosomal instability (CIN), microsatellite instability (MSI), consensus molecular subtypes (CMS), or a CpG Island Methylator Phenotype (CIMP);

Hepatocellular carcinoma (HCC) subtypes, including steatohepatitic, clear cell, macrotrabecular-massive, scirrhous, chromophobe, fibrolamellar, neutril-rich, and lympocyte-rich HCCs; and

Non-Small Cell Lung Cancer (NSCLC) subtypes, including adenocarcinoma, squamous cell carcinoma, and large cell carcinoma.

In one embodiment, given the large number of features (e.g., hundreds of thousands) in the RNA count input, and relatively small size of cancer dataset, the number of features may sometimes exceed the number of samples in a single dataset. Thus, multiple datasets from different data sources are often used at training. The AI-based cancer detection and subtyping tool uses variational Bayes inference and semi-supervised training to adjust for batch effects, learn a low dimensional distribution explaining biological variability of the data, and classify cancer state, tissue of origin, cancer subtype, and/or other aspects of a detected cancer as described above. For example, the VAE may translate oncRNA training data into a latent space such that batch effects due to training data from different suppliers, data sources, and/or other non-biological variations may be reduced or removed. The AI-based cancer detection and subtyping tool may thus optimize its parameters through learning a statistical representation of the input dataset through its variational Bayes objectives through a two-phase training process. Therefore, the VAE-based AI-based cancer detection and subtyping tool models the small RNA sequence read counts in the serum while removing batch effects resulting from using two or more suppliers, two or more data sources, and/or other known or unknown sources of variation.

In this way, the AI-based cancer detection and subtyping tool may be trained on a large aggregated dataset composed of multiple smaller datasets that do not necessarily need to be the same. The AI-based cancer detection and subtyping tool can thus effectively combine and distill the information in all sub-datasets and learn complex, non-linear relationships between input features (including oncRNAs) and the targets (tumor presence, subtype, etc.), and adjust for unknown/unobserved sources of variation in the data. All these are achieved through a customized semi-supervised deep learning model, which uses the principles of variational Bayes to learn the statistical representation of the data, account for unknown sources of variation, model non-linear dependencies among independent variables, and provide more accurate predictions. This allows combining data from multiple batches or even multiple suppliers, with different data collection and processing protocols, hence enabling the building of large, AI models on the combined dataset.

As the AI model is trained on a large, heterogeneous dataset, it can generalize better than traditional linear or other simpler models. For example, on a Non-Small Cell Lung Cancer (NSCLC) dataset composed of three different batches from two suppliers, traditional linear regression models achieve: Area Under the Curve (AUC): 0.85, Stage I Sensitivity at 95% Specificity: 36%, Adenocarcinoma vs Squamous Cell Carcinoma subtyping sensitivity for late stage (III/IV) at 70% specificity: 46%. The AI-based cancer detection and subtyping tool described herein outperforms traditional linear models with: AUC: 0.98, Stage I Sensitivity at 95% Specificity: 85%, Adenocarcinoma vs Squamous Cell Carcinoma subtyping sensitivity for late stage (III/IV) at 70% specificity: 0.67.

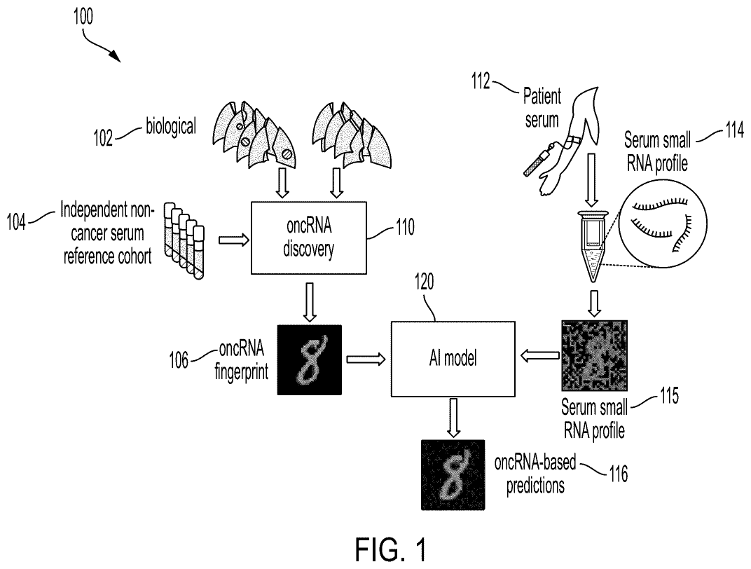

FIG. 1 is a simplified diagram illustrating a process 100 of using an AI-based diagnostic platform for cancer detection and subtyping, according to some embodiments described herein. Process 100 illustrates a liquid biopsy approach for cancer detection using newly annotated lung cancer-emergent and tumor-released oncRNAs as a signature for cancer detection from blood.

In one embodiment, biological samples 102 such as NSCLC and tumor adjacent normal samples from a public dataset such as The Cancer Genome Atlas (TCGA) tissue datasets may be input to a oncRNA discovery module 110 for oncRNA selection. For example, to identify a set of oncRNAs, smRNA-sequencing data from 10,403 tumor and 679 adjacent normal tissue samples from TCGA spanning 32 unique tissue types may be collected. Quality control was applied to the GRCh38-aligned BAM files to remove reads that were <15 base pairs or were considered low complexity based on a DUST score>2. Additionally, reads that mapped to chrUn, chrMT, or other non-human transcripts are removed. After filtering, de novo smRNA loci are identified by merging all reads across the 11,082 TCGA samples and performing peak calling on the genomic coverage to identify a set of smRNA loci that were <200 base pairs. This resulted in 74 million distinct candidate loci for feature discovery.

In another example, for discovery of lung tumor-specific oncRNAs, lung tumors (n=999) and all adjacent normals (n=679) are focused on and filtered the candidate loci for those that appeared in at least 1% of samples resulting in 1,293,892 smRNAs. A generalized linear regression model may identify those smRNAs that were significantly more abundant in lung tumors compared to normal tissues. Such model can be adjusted for age, sex, and principal components to capture the global smRNA expression variability across tissues and batches. After multi-testing correction suggestively significant smRNA features (FDR q<0.1) that were enriched in lung tumors (OR>1) resulting in ˜260 k lung-tumor associated oncRNAs are obtained for downstream applications in serum.

In one embodiment, the TCGA smRNA-seq database to identify 255,393 NSCLC-specific oncRNAs through differential expression analysis of NSCLC and non-cancerous tissues. NSCLC oncRNA fingerprints 106 may be generated from TCGA NSCLC and tumor adjacent normal samples 102 and a set of independent non-cancer serum reference cohort 104. The oncRNA fingerprint 106 may be input to an AI model 120 that identifies scarce smRNAs that are selectively expressed in lung tumors versus normal lung tissues.

In one embodiment, serum smRNA data 115 may be generated from an in-house dataset of patient serum 112. For example, patient serum 112 may be collected from 1,050 treatment naive individuals (419 with NSCLC and 631 without history of cancer). These samples are sourced from two different suppliers, where each supplier provided both cancer and control samples as shown in Table 1 below. Cell-free smRNA may be isolated from 0.5 mL of serum to quantify the expression of NSCLC-specific oncRNAs identified in the TCGA data. A total of 237,928 (93.15%) of the selected oncRNAs from tissue samples were detected in at least one of the serum samples.

TABLE 1
Sample demographics. Sample size and key demographic
aspects of training set and held-out validation set.

| | | Training set | | Validation set | |
|---|---|---|---|---|---|
| Demographics | | Control | Cancer | Control | Cancer |
| Sample size | Count, n | 506 | 334 | 125 | 85 |
| Age | Mean (SD) | 62.18 | 65.84 | 61.80 | 63.85 |
| | | (11.75) | (9.60) | (10.80) | (10.35) |
| Sex | Female (%) | 238 | 125 | 50 | 40 |
| | | (47.04%) | (37.43%) | (40.00%) | (47.06%) |
| Smoking | Never-Smoked, n | 271 | 34 | 71 | 7 |
| status | (%) | (53.56%) | (10.18%) | (56.80%) | (8.24%) |
| BMI | BMI obese (≥30), | 124 | 72 | 28 | 15 |
| | n (%) | (24.51%) | (21.56%) | (22.40%) | (17.65%) |
| Race | White, n (%) | 253 | 220 | 62 | 55 |
| | | (50.00%) | (65.87%) | (49.60%) | (64.71%) |
| | Black/African | 54 | 12 | 14 | 1 |
| | American, n (%) | (10.67%) | (3.59%) | (11.20%) | (1.18%) |
| | Asian, n (%) | 15 | 4 | 3 | 0 |
| | | (2.96%) | (1.20%) | (2.40%) | (0.00%) |
| | Other/Unknown, n | 184 | 98 | 46 | 29 |
| | (%) | (36.36%) | (29.34%) | (36.80%) | (34.12%) |
| Ethnicity | Hispanic, n (%) | 179 | 12 | 46 | 5 |
| | | (35.38%) | (3.59%) | (36.80%) | (5.88%) |
| | Non-hispanic, n | 281 | 316 | 59 | 80 |
| | (%) | (55.53%) | (94.61%) | (47.20%) | (94.12%) |
| | Other/Unknown, n | 45 | 6 | 19 | 0 |
| | (%) | (8.89%) | (1.80%) | (15.20%) | (0.00%) |
| Source | Indivumed, n (%) | 183 | 258 | 46 | 65 |
| | | (36.17%) | (77.25%) | (36.80%) | (76.47%) |
| | MT Group, n (%) | 323 | 76 | 79 | 20 |
| | | (63.83%) | (22.75%) | (63.20%) | (23.53%) |

Thus, serum smRNA profiles 114 may be extracted from a set of patient serums 112. Such serum smRNA profiles 115, together with the oncRNA fingerprints 106, may then be input to the AI model 120 to train the AI model 120 to identify cancer related oncRNA features. For example, given an input of oncRNA profile (count data), AI model 120 may generate oncRNA-based predictions 116, which may indicate one or more of a cancer diagnose (whether there is cancer detected), a tissue of origin, a cancer subtype, and/or the like.

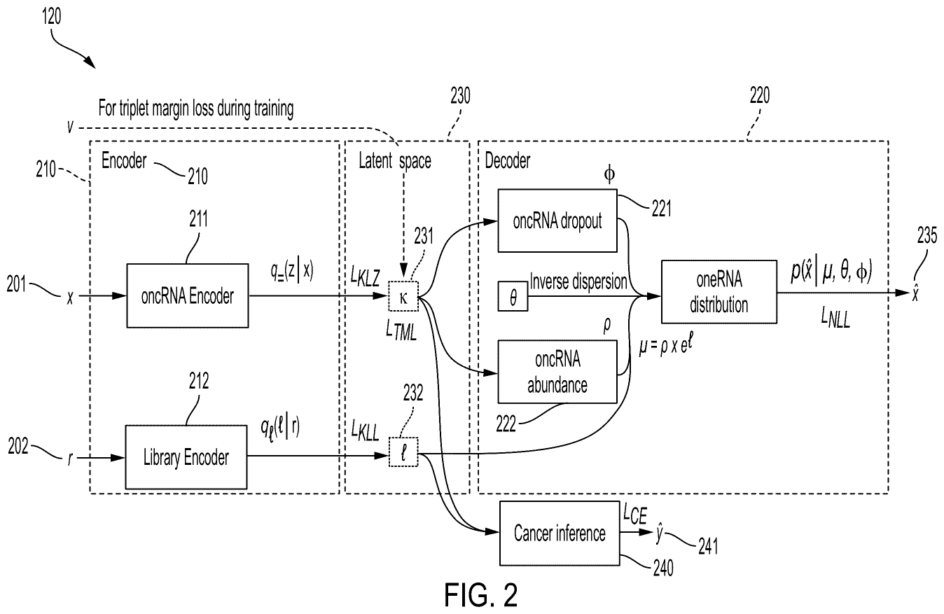

FIG. 2 is a simplified diagram illustrating an example architecture of the neural network model 120 described in FIG. 1, according to embodiments described herein. AI model 120 may comprise a neural network model of a customized, regularized, multi-input, and semi-supervised variational auto encoder (VAE) 210, and a decoder 220.

In one embodiment, given an oncRNA count matrix generated from a training sample (e.g., from NSCLC oncRDN fingerprint 106 and/or serum smRNA profile 115 in FIG. 1), let xᵢ∈Z^{d} (201) denote and ᵣ∈Z^{m} (202) denote counts for doncRNAs and m endogenous highly-expressed smRNAs for the i-th sample, respectively. And let yᵢ∈{0, 1}^{b}×R^{t} and vᵢ∈Z^{c} denote the b binary and/real targets (cancer status) and the c known confounders (sample source, processing batch, etc), respectively.

In one embodiment, an oncRNA encoder 211 may encode oncRNA count data x which was originally in a high-dimensional space into a low-dimensional latent variable Z (231) in the latent space 230, using a mapping ƒ_{z}: X→Z (called oncRNA encoder 211). The oncRNA encoder 211 captures characteristics of variation in X 201.

In one embodiment, because a common source of variation in transcriptomic data originates from the total sequenced RNA, an oncRNA might not be observed for two reasons: either it does not exist and is not secreted or it is indeed in blood but due to low-volume blood sampling or limited sequencing, it has not been picked up in the experiment. Thus, an additional encoder, referred to as the library encoder 212 may encode a set of endogenous highly-expressed RNAs r 202: [symbol] : R→ [symbol] to compute a normal distribution [symbol] ( [symbol] |r) as a proxy for the log of library size. The encoded variable [symbol] ∈R (232) may represent another unobserved random variable that accounts for input RNA level and library sequencing depth. In other words, the library size is log-normal with priors originating from the log of mean and variance of mrᵢ in a given min-batch. As a result, [symbol] (232) shows a strong correlation with the total number of oncRNA reads, even though it is not derived from oncRNAs.

For example, oncRNA encoder 211 may comprise one hidden layer for encoding oncRNAs with 1,500 hidden units, while library encoder 212 may compri [symbol] one hidden layer for encoding library size from endogenous RNAs with 1,500 units. The latent space 230 may comprise an embedding space of d=50 latent variables for learning the Gaussian distribution underlying the oncRNA data, an embedding space of s =1 latent variable for learning the library size distribution from endogenous RNAs.

In one embodiment, decoder 220 may adopt another mapping g: Z→X such that decoder output 235 x\hat{ }=g(z)=g(ƒ(x)) is approximately the same as input x (201), e.g., ∥x−x∥² is small. In variational auto-encoders instead of deterministically mapping x to z, x 201 is mapped to a (usually Gaussian) distribution q_{z}(z|x). When reconstructing x, z 231 may be [symbol] sampled from the distribution q_{z}(z|x) and using this sample, a distribution for reconstructed x as x\hat{ }=pₓ(x|z) may be generated.

In one embodiment, decoder 220 may comprise an oncRNA dropout module 221, and an oncRNA abundance module 222. For example, similar to gene counts across cells in single-cell RNA-seq data, any oncRNA is observed in only a few samples and its counts are mostly zeros, also called zero-inflated. If assuming the non-zero counts follow a negative binomial distribution, the oncRNAs count may be represented as a conditional zero-inflated negative binomial (ZINB) distribution p (x|z, [symbol] ), where z∈R^{k}, k«d is the latent embedding of x. Thus, the oncRNA dropout module 221 may generate zero-inflation parameter ϕᵢ: ƒ_{ϕ}: Z→ϕ and the oncRNA abundance module 222 may generate transcription scale parameter ρᵢ through ƒ_{ρ}: Z→ρ, where ƒ_{ρ} involves a soft max step, enforcing representation of the expression of each oncRNA as a fraction of all expressed oncRNAs.

In one embodiment, the Gamma-Poisson representation of the negative binomial distribution, μ=ρᵢ× [symbol] , may provide the shape parameter of the Gamma distribution, and input-independent learnable parameter θ will represent the inverse dispersion. oncRNA distribution conditioned on the parameters μ, θ and ϕ can thus be generated.

During training, given training input x 201 and r 202, oncRNA encoder 211 and library encoder 212 may generate a low-dimensional Gaussian distributions q_{z}(z|x) and [symbol] ( [symbol] |r), so that zero-inflated negative binomial distribution qₓ(x|z, [symbol] ) has the generative capability of producing realistic in silico oncRNA profiles.

Specifically, a first loss L_{KLZ} may be computed based on oncRND encoder 211 output latent variable distribution, L_{KLZ}=D_{KL}(q_{z}(z|x)∥p(z)), where D_{KL} is the Kullback-Leibler divergence and p(z)=N(0, I) is the prior distribution for z.

A second loss L_{KLL} may be computed based on library encoder 212 output latent variable distribution, L_{KLL}=D_{KL}( [symbol] ( [symbol] |r)∥p( [symbol] |r)), where p( [symbol] |r) is the prior log-normal distribution for [symbol] . Unlike z, the prior distribution for [symbol] is different from batch to batch and its log-mean and log-standard deviation are computed based on values of r in each mini-batch B.

A third loss, which may be a reconstruction loss, may be computed as the negative log likelihood of a zero-inflated negative binomial distribution describing the distribution of the input oncRNA data: L_{NLL}=−Σᵢ log pₓ(xᵢ|μᵢ, θᵢ, ∅ᵢ), where μᵢ is the product of the soft max of ƒₚ (representing transcription scale of each oncRNA) and [symbol] i ; and θᵢ, ϕᵢ represent inverse dispersion and zero-inflation probability, respectively.

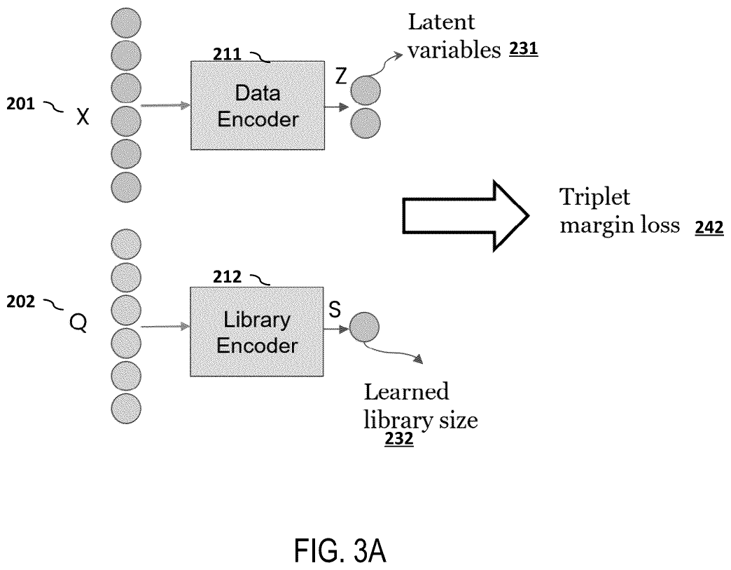

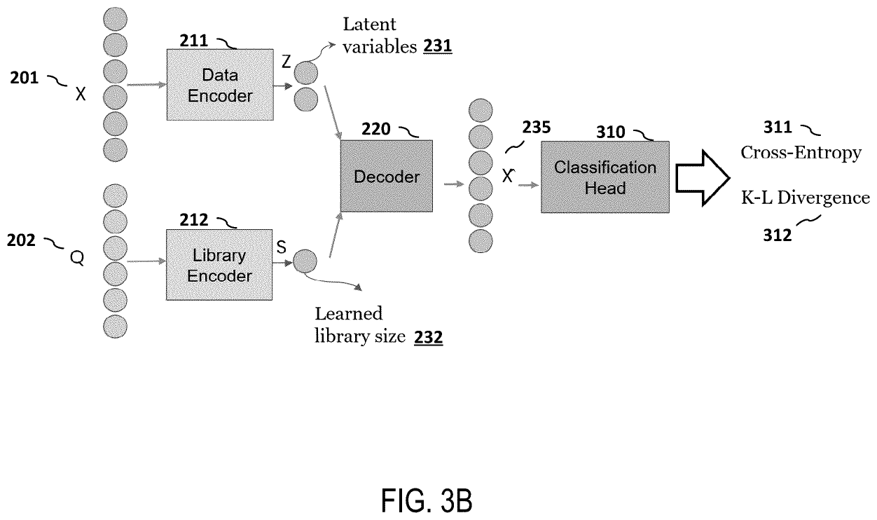

A fourth loss, which may be a contrastive loss, or a triplet margin loss, may be computed using known confounders v (from annotations in training batch) on z:

$$\\ {L}_{TML} = max ( 0 , d ( z , p ) - d ( z , n ) + α )$$

where p and n are “positive” and “negative” samples corresponding to a sample z, as further described in FIG. 3A. The triplet margin loss may force all the cancer samples from different sources projected in the proximity of each other in the latent space 230. By minimizing the triplet margin loss during training, the distance between samples that have the same label (e.g., all cancer samples or all control samples) but are from a different confounder group (e.g. source, supplier, etc.) in the oncRNA embedding space is minimized, while the distance between samples that have different labels are maximized.

A fifth loss, which may be computed as the cross-entropy loss L_{CE} [symbol] etween the predicted sample label 241 and the original sample labels (e.g. cancer vs. control). For example, a cancer inference module 240 may generate the predicted label 241 ŷ from latent variables z 231 and l 232.

In one embodiment, during training, one or more of the five losses may be minimized during backpropagation of the encoder 210 and/or decoder 220 to update the weights. For example, as further illustrated in relation to FIGS. 3A-3B, the different losses may be used to update the encoder 210 and/or decoder 220 in one or more different training phases. In one implementation, a joint training loss may be computed as the weighted summation of these five losses:

$$\\ L = {λ}_{1} {L}_{KLZ} + {λ}_{2} {L}_{KLL} + {λ}_{3} {L}_{NLL} + {λ}_{4} {L}_{TML} + {λ}_{5} {L}_{CE}$$

The encoder 210 and decoder 220 may then be updated by the joint loss L via backpropagation. Additional details of backpropagation of a neural network model to update weights of the neural network model may be discussed in FIG. ??.

In this way, the semi-supervised training framework of AI model 120 using the five different types of losses allows its representation learning to capture the biological signal of interest (e.g. cancer detection) while removing unwanted confounders (such as batch effects).

FIGS. 3A-3B provide simplified block diagrams illustrating example aspects of a two-phase training process of the AI-based cancer detection and subtyping tool, according to embodiments described herein. For example, two types of RNA count data are used as training input, X (201 in FIG. 2, RNA biotypes used for classification) and Q (202 in FIG. 2, endogenous highly expressed RNA biotypes used only for estimating the dataset library size as a latent variable).

For example, the training data may be a combination of smaller datasets of RNA biotypes from different data sources, vendors, or other suppliers. The training data may comprise RNA input counts that are counts of certain RNA sequences previously established as oncRNAs. The training input sample, X or S, may take a form of oncRNA count annotated with corresponding information such as one or more labels of: the presence of a tumor (Yes/No), a size, lymph node invasion and metastasis state (TNM), a subtype of the tumor (e.g., adenocarcinoma or squamous cell carcinoma, etc.), a tissue of origin (e.g., lung, etc.), a gene expression profile of the tumor, treatment planning and monitoring, predicted minimal residual disease (MRD), and/or the like.

For example, data encoder 211 and/or library encoder 212 may have a hidden layer of size 1,500 and map X to parameters of z_{d} with 50 dimensions and map Q to parameters of zₛ with 1 dimension. Decoder 220 may have one hidden layer for decoding oncRNA data from the latent distribution. A dropout rate (p=0.5), L₂ regularization (L₂=2) may be adopted. The classification layer 310 may have one hidden layer of size 25, mapping the 50 normalized latent values to generative predictions for each class.

FIG. 3A shows a representative aspect of a first training phase using a triplet margin loss 242. To allow the AI-based cancer detection and subtyping tool to learn biological representations of the data irrespective of supplier, dataset, and other technical variations, the distance metric learning may be used. Similar to that described in FIG. 2, training samples X 201 and Q 202 are encoded by the data encoder 211 and library encoder 212, respectively, into latent variables 231 and library latent variable 232. The data encoder 211 or the library encoder 212 may be a VAE that projects the original RNA biotypes X 201 and Q 202 as latent variables Z and a latent library size S in the latent space.

In one embodiment, for each training sample indexe [symbol] as i, ω triplets may be sampled for each confounder v^{c} as follows: first, randomly picking a “positive” anchor j≠i, such that training sample i and j share the same classification label: yᵢ=yⱼ, but do not share the same confounder vᵢ^{c}≠vⱼ^{c}; next, randomly picking a “negative” anchor j′≠i, such that training sample i and j′ do not share the same classification label: yᵢ≠yⱼ, and do not share the same confounder vᵢ^{c}≠vⱼ^{c}; next the samples (i, j, j′) are added to Tᵢ, the set of triplets for i. At the end of this sampling process, each sample will have |Tᵢ|=ω×c triplets picked for it, where ω is a hyperparameter set to a pre-defined number (e.g., 8, 16, 18, etc.).

In one example, the sampled triplets of training samples may be encoded by data encoder 211 and/or library encoder 212 into latent variables to compute the triplet margin loss 242:

$$\\ {L}_{TML} = max ( 0 , d ( z , p ) - d ( z , n ) + α )$$

where p represents the “positive” sample for training sample z, and n represents the “negative” anchor for training sample z; α is a hyperparameter that enforces what should be the minimum difference of distances between a sample and its positive and negative anchors in the latent space, and it is set to α=1.

In this way, triplet margin loss 242 is computed based on the latent variables to train the VAE data encoder 211 and the VAE library encoder 212, showing positive anchors for which the model should minimize each sample's distance in the embedding space, and negative anchors for which the model should maximize their distance in the embedding space. In other words, if healthy control samples are from sources A and B, and cancer samples are from sources C and D, the VAE is trained such that it projects input samples from data sources A and B close to each other but far from C and D samples in the embedding space. Similarly, a contrastive loss may be computed based on the latent representations to train the VAE.

During training a cost function may be added such that moving samples from different sources or processing batches that share the same label (e.g. cancer samples from different sources) closer to each other, while moving samples with different labels (e.g. cancer samples from non-cancer samples) further apart.

FIG. 3B shows a representative aspect of a second training phase using a cross-entropy loss 311 and a K-L divergence loss 312. In one embodiment, following a first training phase using the triplet margin loss 242 described in relation to FIG. 3A, at a second training phase, the trained VAE data encoder 211 and the VAE library encoder 212 from the first training phase may be connected to one or more decoder heads 220, which in turn generate a reconstructed RNA biotype 235 used for classification X′ from the latent variables Z and S. A classification head 310 may be attached to predict output distribution, e.g., a presence of a tumor, tissue of origin, subtype of the tumor, and/or the like. A loss may be computed based on the predicted output distribution compared with the ground-truth labels of presence of a tumor, tissue of origin, subtype of the tumor corresponding to the input sample from the training dataset. Such loss may be a cross-entropy loss 311 (for the classification head) or Kullback-Leibler (K-L) divergence loss 312. The VAE encoders (e.g., the data encoder 211 and the library encoder 212) and the respective decoder head 220 may then be jointly updated based on a combination of the cross-entropy loss 311 and K-L loss 312.

In one embodiment, the VAE encoders and the respective decoder head may be separately trained in two different training phases as shown in FIGS. 1A-1B.

In another embodiment, the VAE encoders and the respective decoder head may be jointly trained based on a combined loss of the cross-entropy loss, the KL divergence and the triplet margin loss.

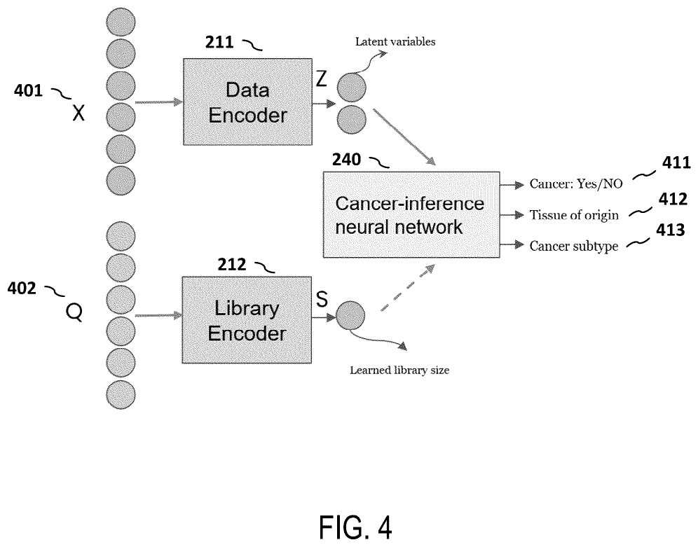

FIG. 4 provides a simplified block diagram illustrating representative aspects of the inference/testing process of the AI-based cancer detection and subtyping tool, according to embodiments described herein. At the inference/testing stage, the AI-based cancer detection and subtyping tool comprising the trained VAE and a cancer-inference neural network model (e.g., various classification heads for predicting/classification of different outputs), may, in response to an input of oncRNA count obtained from any biofluid or tissue samples, generate a predicted output. Depending on the types of classification heads in the cancer-inference neural network model, the output may comprise such as, but not limited to detection of the presence of a tumor 411, a predicted size, lymph node invasion and metastasis state (TNM), a predicted subtype of the tumor 413, a predicted tissue of origin 412, a predicted gene expression profile of the tumor, predicted treatment planning and monitoring, predicted minimal residual disease (MRD), and/or the like.

For example, the cancer-inference neural network model 240 performs classification through a 2-layer perceptron head. The input of the classification head comes from the batch-normalized product of oncRNAs and library size embeddings (e.g., 231, 232 in FIG. 2), i.e., z× [symbol] . During training, latent variables are sampled from q_{z}(z|x) η=100 times for each data point to improve model robustness and sensitivity to noise. At inference, the deterministic expected values of z and [symbol] are input to the classification head.

As shown in FIG. 4, the cancer-inference neural network model 240 may comprise different classification heads for generating a probability distribution of a presence of cancer 411, e.g., P (presence of cancer=Yes|oncRNA count). A threshold may be applied such that a prediction of a presence of cancer may be output when P (presence of cancer=Yes|oncRNA count)>Th.

As another example, the cancer-inference neural network model 240 may generate a probability distribution of a tissue of origin 412, e.g., distribution among a pre-defined set of possible tissues of origin such as {lung, not lung}, etc. In one implementation, an arg max operation may be applied on the probability distribution to output one or more predicted tissue(s) of origin.

As another example, the cancer-inference neural network model 240 may generate a probability distribution of cancer subtypes 413, e.g., distribution among a pre-defined set of possible cancer subtypes such as {adenocarcinoma, squamous cell carcinoma, null} with the class “null” corresponding to the case when there is no cancer, etc. In one implementation, an arg max operation may be applied on the probability distribution to output predicted cancer subtype.

Similarly, the cancer-inference neural network model 240 may generate a probability distribution of TNM stage of the cancer, and/or recommendation of treatments.

In one embodiment, the cancer-inference neural network model 240 may generate the output predictions in a joint output vector, e.g., {presence of cancer, tissue of origin, subtype}. For example, the predicted presence of cancer, tissues of origin and/or subtype may be generated by one or more classification heads in parallel. Example output vectors may take a form as {Yes (cancer), lung, adenocarcinoma}, {Yes (cancer), lung, squamous cell carcinoma}, {No (cancer), null, null}, {Yes (cancer), not lung, null}, and/or the like.

In one embodiment, the cancer-inference neural network model 240 may generate the output predictions in a progressive fashion. For example, the cancer-inference neural network model may first determine a presence of cancer. If the presence of cancer is determined, the cancer-inference neural network model may employ other classification heads to generate a predicted cancer subtype, tissue of origin, and/or the TNM stage of the cancer.

The testing/inference stage described in FIG. 4 may be applied in cancer diagnostics from an RNA sample. For example, in one embodiment, a cancer diagnostic method may include receiving and processing an RNA sample to identify small RNAs such as oncRNAs. Representative embodiments of detection and/or quantification of oncRNA molecules in a sample of the subject may be found in PCT International Application Pub. WO 2022/040106. The cancer diagnostic method further includes feeding the detected small ncRNA data, e.g., the oncRNA count, grouped as the input data X (oncRNA count for classification) and Q (endogenous highly expressed oncRNA for library size estimation), into the data encoder and library encoder shown in FIG. 2, respectively. The cancer-inference neural network model may in turn generate a probability distribution indicating the likelihood of cancer, and/or a probability distribution indicating the likelihood of a particular tissue of origin or cancer subtype.

In another example, the testing/inference stage described in FIG. 4 may be applied in cancer diagnostics from a biological sample (e.g., a blood sample, etc.) from a subject suspected of cancer. A cancer diagnostic method may include receiving and processing a biological sample from a subject at a lab setting. For example, the expression level of one or more small non-coding RNA biomarkers may be determined in a biological sample derived from a subject. A sample derived from a subject is one that originates from a subject. Such a sample may be further processed after it is obtained from the subject. For example, RNA may be isolated from a sample. In this example, the RNA isolated from the sample is also a sample derived from a subject. A biological sample useful for determining the level of one or more small non-coding RNA biomarkers may be obtained from essentially any source, including cells, tissues, and fluids throughout the body.

In some embodiments, the biological sample used for determining the level of one or more small non-coding RNA biomarkers is a sample containing circulating small ncRNAs, e.g., extracellular small ncRNAs. Extracellular small ncRNAs freely circulate in a wide range of biological materials, including bodily fluids, such as fluids from the circulatory system, e.g., a blood sample or a lymph sample, or from another bodily fluid such as urine or saliva. Accordingly, in some embodiments, the biological sample used for determining the level of one or more small ncRNA biomarkers is a bodily fluid, for example, blood, fractions thereof, serum, plasma, urine, saliva, tears, sweat, semen, vaginal secretions, lymph, bronchial secretions, CSF, whole blood, stool, interstitial fluid, synovial fluid, gastric acid, sebum, mucus, bile, etc. In some embodiments, the sample is a sample that is obtained non-invasively, such as a stool sample. In some embodiments, the sample is a serum sample from a human.

The substance may be solid, for example, a biological tissue. The substance may comprise normal healthy tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substance may comprise a tumor. Tumors may be benign (non-cancer), pre-malignant, or malignant (cancer), or any metastases thereof. The substances may comprise a mix of normal healthy tissues or tumor tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

In some embodiments, the substance may comprise a variety of cells, including: eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, and circulating cells in the human blood. In some embodiments, the substance may comprise contents of a cell, such as, for example, the contents of a single cell or the contents of multiple cells.

In some embodiments, any of the methods disclosed herein comprise using a small volume sample. In some embodiments, the methods disclosed comprise isolating total RNA or small RNA, e.g., small non-coding RNA, and/or amplifying total or small RNA in a sample of no more than about 20 microliters of sample, 40 microliters of sample, 80 microliters of sample, 100 microliters of sample, 200 microliters of sample, 300 microliters of sample, 400 microliters of sample, 500 microliters of sample, 600 microliters of sample, 700 microliters of sample, 800 microliters of sample, 900 microliters of sample, 1 milliliter of sample, 1.1 milliliters of sample, 1.2 milliliters of sample, 1.3 milliliters of sample, 1.4 milliliters of sample, 1.5 milliliters of sample, 1.6 milliliters of sample, 1.7 milliliters of sample, 1.8 milliliters of sample, 1.9 milliliters of sample, 2.0 milliliters of sample. In some embodiments, the sample size is from about 25 microliters to about 2 milliliters of liquid sample in the form of subject plasma, whole blood or serum.

In some embodiments, the methods disclosed comprise isolating total RNA and/or amplifying non-coding RNA in a sample of no more than about 20 microliters of serum, 40 microliters of serum, 80 microliters of serum, 100 microliters of serum, 200 microliters of serum, 300 microliters of serum, 400 microliters of serum, 500 microliters of serum, 600 microliters of serum, 700 microliters of serum, 800 microliters of serum, 900 microliters of serum, 1 milliliter of serum, 1.1 milliliters of serum, 1.2 milliliters of serum, 1.3 milliliters of serum, 1.4 milliliters of serum, 1.5 milliliters of serum, 1.6 milliliters of serum, 1.7 milliliters of serum, 1.8 milliliters of serum, 1.9 milliliters of serum, 2.0 milliliters of serum.

Circulating small non-coding RNAs include small non-coding RNAs in cells, extracellular small non-coding RNAs in microvesicles, in exosomes and extracellular small non-coding RNAs that are not associated with cells or microvesicles (extracellular, non-vesicular small non-coding RNA). In some embodiments, the biological sample used for determining the level of one or more small non-coding RNA biomarkers (e.g., a sample containing circulating small non-coding RNA) may contain cells. In other embodiments, the biological sample may be free or substantially free of cells (e.g., a serum sample). In some embodiments, a sample containing circulating small non-coding RNAs, e.g., extracellular small non-coding RNAs, is a blood-derived sample. Blood-derived sample types may include, e.g., a plasma sample, a serum sample, a blood sample, etc. In other embodiments, a sample containing circulating small non-coding RNAs is a lymph sample. Circulating small non-coding RNAs are also found in urine and saliva, and biological samples derived from these sources are likewise suitable for determining the level of one or more small non-coding RNA biomarkers.

In some embodiments, any of the methods of the disclosure comprise the operation of isolating total RNA or small RNA from a sample or cell or extracellular vesicle. Methods of isolating RNA for expression analysis from blood, plasma and/or serum (see for example, Tsui NB et al. (2002) Clin. Chem. 48,1647-53, incorporated by reference in its entirety herein) and from urine (see for example, Boom R et al. (1990) J Clin Microbiol. 28, 495-503, incorporated by reference in its entirety herein) have been described.

In some embodiments, biological samples may be subjected to one or more processing operations as part of methods and systems as described herein. A processing operation may be carried out to isolate a substance from the biological sample, to purify the biological sample, to separate the biological sample into one or more fractions for further use or processing, to quantitate the amount of a substance, to detect the presence or absence or one or more substances, to transform or modify a substance for further downstream processing or analysis, or any combination thereof. For example, enzymes may be added to digest protein and remove contamination, or to inactivate nucleases that might otherwise degrade nucleic acids (RNA or DNA) during purification. The one or more processing operations may immediately follow sample collection, may be immediately prior to another processing or assaying operation, or may be carried out contemporaneously with another processing or assaying operation. The processing operation(s) may also be carried out on a sample or an intermediate in the process that has been appropriately stored for a designated amount of time. Any number of suitable processing operations may be performed on the biological sample or part thereof.

Processing operations as described herein may include, but are not limited to, immunoassays, enzyme-linked immunosorbent assays (ELISA), radioimmunoassays (RIA), ligand binding assays, functional assays, enzymatic assays, enzymatic treatments (e.g., with kinases, phosphatases, ligases, transcriptases, reverse transcriptases), enzymatic digestions (e.g., nucleases), spectroscopic assays (e.g., UV-vis spectroscopy, Fourier transform infrared spectroscopy, circular dichroism spectroscopy) spectrophotometric assays (e.g., ultraviolet-visible light spectrophotometry), immunoprecipitations (IP), sequencing reactions, electrophoresis, chromatography, enrichments, pull-downs, and mass spectrometry (MS). In some embodiments, a method as described herein comprises not performing one or more processing operations.

One or more processing operations may be performed on a sample or portion thereof. The methods and systems disclosed herein may comprise performing 1, 2, 3, 4, 5, 6, 7, 8, 9, 10, 11, 12, 13, 14, 15, 16, 17, 18, 19, 20, or more processing operations on a sample or portion thereof. The one or more processing operations may be performed sequentially or simultaneously. The one or more processing may be performed on the same sample or portions (e.g., aliquots, fractions) thereof or they may be performed on different samples.

In some embodiments, biological samples containing or suspected of containing RNAs are subjected to one or more processing operations to facilitate downstream processing operations (e.g., isolation). In some embodiments, a sample containing or suspected of containing RNAs may be treated with one or more enzymes, cofactors, and/or other reagents to bring about an end-repair process. The sample containing or suspected of containing one or more RNAs is subjected to treatment with a polynucleotide kinase (PNK) to ensure that end-modified RNA species having a 3′-phosphate group are not lost to further analysis. That is, PNK enzymes dephosphorylate 3′-phosphate RNA species and thus allow for subsequent polyadenylation and inclusion in downstream processing steps.

In some embodiments, biological samples containing or suspected of containing one or more RNAs are subjected to one or more processing operations to remove or change RNA modifications that may inhibit further downstream processing (e.g., subsequent reverse transcription). Alternatively or additionally, chemical modifications may be removed or retained to determine the presence or absence of a relationship between chemical modifications and a disease state or any pathological state, e.g., a cancer state of a subject or population of subjects. Chemically modified RNA bases may include, without limitation, N⁶-methyladenosine (m⁶A), inosine (I), 5-methylcytosine (m⁵C), pseudouridine (Ψ), 5-hydroxymethylcytosine (hm⁵C), N¹-methyladenosine (m¹A), or 7-methylguanosine (m⁷G). For example, biological samples which contain or are suspected of contained methylated RNAs (e.g, comprising m⁶A, m⁵C, hm⁵C, m¹A, or m⁷G) may be treated with one or more demethylation enzymes (e.g, AlkB) to remove alkyl groups which may interfere with downstream processing (e.g., by reverse transcriptases).

In some embodiments, the biological sample is subjected to one or more immunoprecipitation (IP) reactions, such as an in vitro or in vivo crosslinking and IP reaction (CLIP). The one or more IP reactions may enrich or pull-down one or more substances of interest. In some embodiments, an IP processing operation includes a cross-linking operation to covalently link two or more different substances (e.g., to link protein and DNA or protein and RNA). Immunoprecipitation of the cross-linked substances may provide an indication of biological substances that are associated with one another and/or may be used to enrich for specific substances that are known or suspected to interact with one another. In an example, one or more RNAs of interest are cross-linked to one or more corresponding proteins. IP of the proteins cross-linked with the RNAs allows for subsequent isolation and downstream processing of the RNAs of interest. In another example, an IP reaction may be carried out using antibodies specific for an RNA modification of interest, e.g., an adenosine modification (such as m⁶A, m¹A, alternative polyadenylation, or adenosine-to-inosine RNA editing), a uridine modification (such as conversion to pseudouridine), or other RNA modifications as alluded to above.

In some embodiments, the sample is subjected to one or more isolation operations. Isolation operations may target a general class of molecules (e.g., nucleic acids, such as RNAs) or a specific molecule (e.g., a specific annotated RNA molecule).

In some embodiments, a processing operation may comprise adding (e.g., spiking in) one or more substances. The one or more spike-in substances may be for any suitable purpose, including but not limited to, quality control, enrichment of target species, the act of depleting non-target species, or any combination thereof. In some embodiments, a spike-in substance may comprise a synthetic biomolecule (e.g., nucleic acid, such as an RNA or a modified RNA, i.e., an RNA containing base modifications as alluded to above) corresponding to a target biomolecule. In some embodiments, the spike-in substance may comprise an endogenous or exogenous biomolecule. The spike-in molecule may be selected based on any appropriate property such as abundance or relative abundance, origin, sequence or part thereof, global or local structure (such as secondary or tertiary structure), or any combination thereof. Quantification of a spike-in substance following one or more downstream processing operations may be used for quality control as well as quantity control.

In some embodiments, a processing operation may comprises associating a target biomolecule or set of target biomolecules with one or more unique molecular identifiers (UMIs). UMIs may be used to associate biomolecules and indicate them as being derived from the same sample or part thereof. In an example, UMIs (e.g., nucleic acid barcodes) are assigned to or associated with individual samples or parts thereof. Alternatively or additionally, the UMIs are assigned to or associated with an individual subject. UMIs associated with individual biological samples or subjects may allow for pooling of samples during downstream processing.

Aside from associating biomolecules with particular samples or individuals, UMIs may also allow for downstream process control and absolute quantitation or biomolecules (e.g., nucleic acids, such as small non-coding RNAs). In such cases, a single UMI may correspond to one or substantially one target biomolecule. In an example, target biomolecules derived from a biological sample are tagged with individual UMIs corresponding to individual biomolecules. The UMIs allow quantitation of the corresponding target biomolecules and enable controlling for sequencing artifacts (e.g., PCR duplication).

In some embodiments, the UMIs comprise nucleic acid barcodes which are associated with nucleic acids derived from a biological sample as described herein. The nucleic acid barcodes are attached or otherwise associated with the sample-derived nucleic acids to give a set of tagged nucleic acid constructs. In some embodiments, the target nucleic acids are associated with nucleic acid barcodes corresponding to an individual sample. In some embodiments, the target nucleic acids are associated with nucleic acid barcodes corresponding to individual molecules.

One subset of processing operations may serve a different function than another subset of processing operations. For example, one processing operation may be performed to determine the presence of a substance in a sample and a second assay may be performed to isolate the substance from the sample. Processing operations may be performed in any appropriate order. For example, a sample may first be processed to determine the presence of a target substance. A second processing operation may then be used to isolate the target substance from the sample. Optionally, a third processing operation may be performed to purify the isolated substance. In another example, a sample is first processed to isolate a substance or plurality of substances. A second processing operation is then performed on the isolate to determine the presence or absence of a target substance or substances in the isolate. Optionally, a third processing operation is performed between the first and second processing operations to purify the one or more target substances. In another example, a processing operation is performed to enrich for the presence or one or more RNAs in a sample, for instance by enriching DNA made from the RNA, wherein the enrichment is typically carried out using a solid support (e.g., streptavidin beads) that specifically binds to the DNAs of interest (which may, for instance, be modified so as to have a covalently bound biotin group or bound to a complementary nucleic acid sequence that is biotinylated). Before or after the enrichment operation, another processing operation is optionally performed to determine the presence of the one or more RNAs. Still other combinations and sequences of processing operations on a sample are contemplated herein.

In some embodiments, a sample is processed or analyzed by a third party. For example, one party may conduct a purification or enrichment processing operation on a sample. The purified or enriched sample may then be subjected to a subsequent processing operation (e.g., a quantitation) by a different party.

After processing the biological sample to detect small ncRNAs such as oncRNA counts, the cancer diagnostic method further includes feeding the detected small ncRNA data, e.g., the oncRNA count, grouped as the input data X (oncRNA count for classification) and Q (endogenous highly expressed oncRNA for library size estimation), into the data encoder and library encoder shown in FIG. 2, respectively. The cancer-inference neural network model may in turn generate a probability distribution indicating the likelihood of cancer, and/or a probability distribution indicating the likelihood of tissue of origin or cancer subtypes, and/or a predicted treatment.

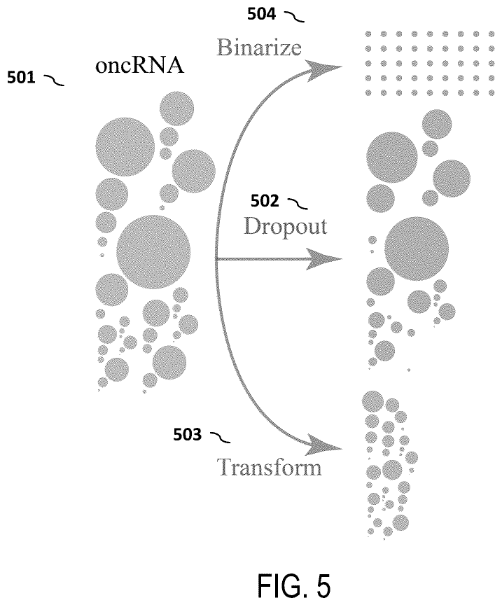

In another embodiment, at the inference stage shown in FIG. 5, the input oncRNA count to the data encoder and/or the library encoder is obtained as counts of certain RNA sequences previously established as oncRNAs.

FIG. 5 provides a simplified block diagram illustrating the data augmentation of RNA data from a training dataset, according to embodiments described herein. During training, original RNA training data 501 such as samples of oncRNA count data, RNA subtypes, and/or the like, is augmented as part of training. For example, specific RNAs, e.g., the counts of specific RNA sequences, are emphasized (binarization 504). Dependency of the model on a group of RNAs is enforced rather than individual RNAs through dropout 502 of some RNAs. Stochasticity of the model is also reduced upon changes in data distribution, e.g., by transforming 503 oncRNA data points to latent variables. Therefore, compared to traditional linear regression models that often fail to handle batch effects, the small RNA sequence read counts in the serum may be accurately modeled while removing batch effects according to supplier, data source, and other unknown sources of variation.

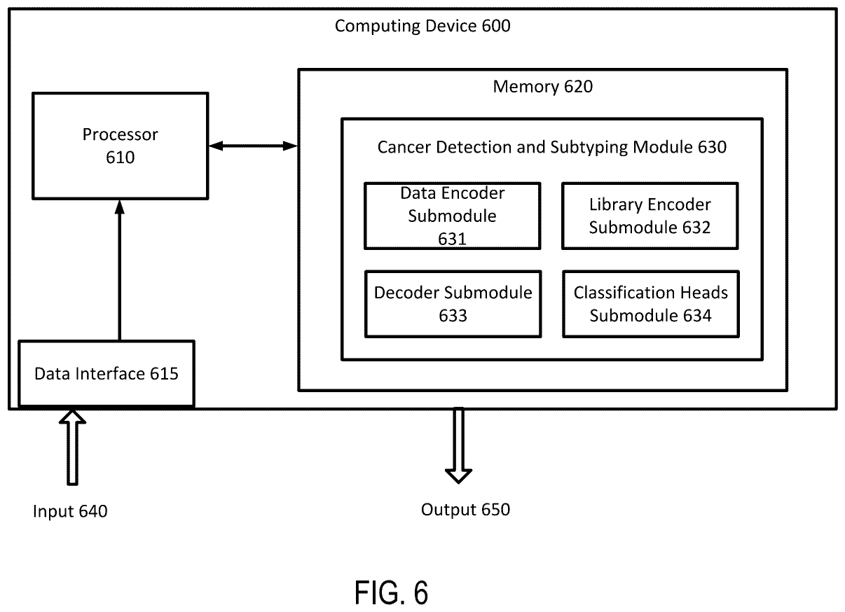

FIG. 6 is a simplified diagram illustrating a computing device implementing an AI-based cancer detection and subtyping module, according to one embodiment described herein. As shown in FIG. 6, computing device 600 includes a processor 610 coupled to memory 620. Operation of computing device 600 is controlled by processor 610. Although computing device 600 is shown with only one processor 610, it is understood that processor 610 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in computing device 600. Computing device 600 may be implemented as a stand-alone subsystem, as a board added to a computing device, and/or as a virtual machine.

Memory 620 may be used to store software executed by computing device 600 and/or one or more data structures used during operation of computing device 600. Memory 620 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Processor 610 and/or memory 620 may be arranged in any suitable physical arrangement. In some embodiments, processor 610 and/or memory 620 may be implemented on a same board, in a same package (e.g., system-in-package), on a same chip (e.g., system-on-chip), and/or the like. In some embodiments, processor 610 and/or memory 620 may include distributed, virtualized, and/or containerized computing resources. Consistent with such embodiments, processor 610 and/or memory 620 may be located in one or more data centers and/or cloud computing facilities.

In some examples, memory 620 may include non-transitory, tangible, machine readable media that includes executable code that when run by one or more processors (e.g., processor 610) may cause the one or more processors to perform the methods described in further detail herein. For example, as shown, memory 620 includes instructions for cancer detection and subtyping module 630 that may be used to implement and/or emulate the systems and models, and/or to implement any of the methods described further herein. cancer detection and subtyping module 630 may receive input 640 such as an input training data (e.g., training oncRNA data) via the data interface 615 and generate an output 650 which may be a predicted distribution relating to a detection of cancer, tissue of origin and/or subtype, and/or a predicted treatment.

The data interface 615 may comprise a communication interface, a user interface (such as a voice input interface, a graphical user interface, and/or the like). For example, the computing device 600 may receive the input 640 (such as a training dataset) from a networked database via a communication interface. Alternatively, the computing device 600 may receive the input 640, such as oncRNA count, from a user via the user interface.

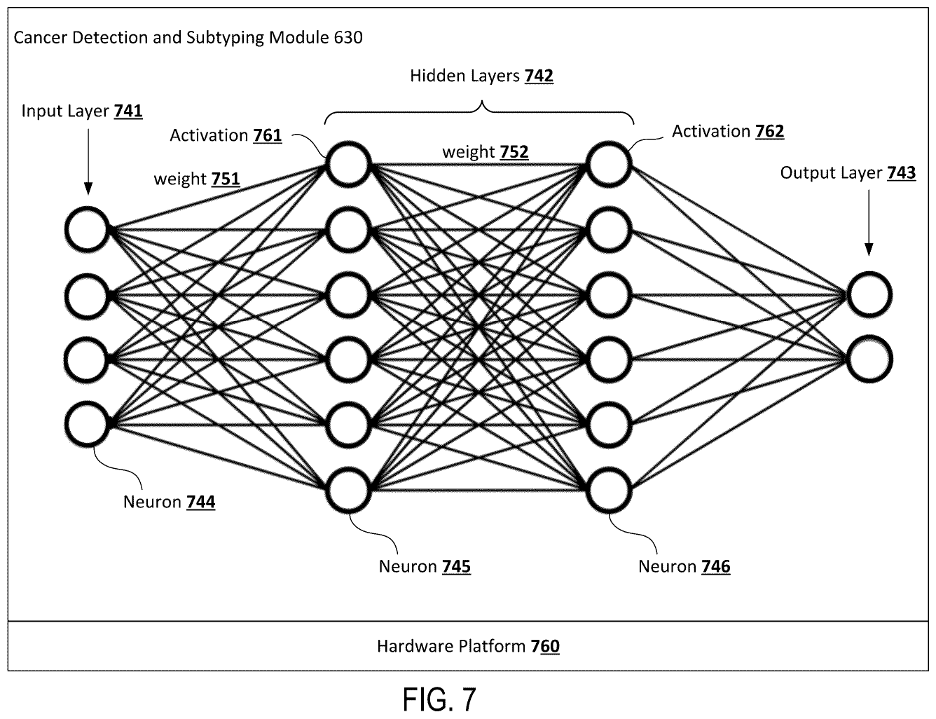

In some embodiments, the cancer detection and subtyping module 630 is configured to predict the presence of cancer, its tissue of origin and/or subtypes in response to an input oncRNA count sample as described herein. The cancer detection and subtyping module 630 may further include a data encoder submodule 631 (e.g., 211 in FIGS. 2, 3A-3B and 4), a library encoder submodule 632 (e.g., 212 in FIGS. 2, 3A-3B and 4), a decoder submodule 633 (e.g., 220 in FIGS. 2 and 4) and one or more classification heads 634 (e.g., 240 in FIG. 2 and 4, or 310 in FIG. 3B). These submodules 631-634 may each take a structure of a neural network as described in FIG. 7.

In one embodiment, the cancer detection and subtyping module 630 and its submodules 631-634 may be implemented by hardware, software and/or a combination thereof.

In one embodiment, the cancer detection and subtyping module 630 and one or more of its submodules 631-434 may be implemented via an artificial neural network. The neural network comprises a computing system that is built on a collection of connected units or nodes, referred to as neurons. Each neuron receives an input signal and then generates an output by a non-linear transformation of the input signal. Neurons are often connected by edges, and an adjustable weight is often associated with the edge. The neurons are often aggregated into layers such that different layers may perform different transformations on the respective input and output transformed input data onto the next layer. Therefore, the neural network may be stored at memory 620 as a structure of layers of neurons, including parameters describing the non-linear transformation at each neuron and the weights associated with edges connecting the neurons. An example neural network may be a VAE (e.g., adopted by the data encoder 631 and library encoder 632), a multilayer perceptron (MLP) which may be adopted by the decoder submodule 633, and/or the like.

In one embodiment, the neural network based cancer detection and subtyping module 630 and one or more of its submodules 631-634 may be trained by updating the underlying parameters of the neural network based on the loss described in relation to FIG. 1-5. For example, a loss, such as a triplet margin loss, a cross-entropy loss, a K-L divergence loss, and/or the like as described in relation to the training processes in FIGS. 1A-1B, is a metric that evaluates how far away a neural network model generates a predicted output value from its target output value (also referred to as the “ground-truth” value). Given the loss, the negative gradient of the loss function is computed with respect to each weight of each layer individually. Such negative gradient is computed one layer at a time, iteratively backward from the last layer to the input layer of the neural network. Parameters of the neural network are updated backwardly from the last layer to the input layer (backpropagating) based on the computed negative gradient to minimize the loss. The backpropagation from the last layer to the input layer may be conducted for a number of training samples in a number of training epochs. In this way, parameters of the neural network may be updated in a direction to result in a lesser or minimized loss, indicating the neural network has been trained to generate a predicted output value closer to the target output value.

Some examples of computing devices, such as computing device 600 may include non-transitory, tangible, machine readable media that include executable code that when run by one or more processors (e.g., processor 610) may cause the one or more processors to perform the processes of method. Some common forms of machine-readable media that may include the processes of method are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

FIG. 7 is a simplified diagram illustrating the neural network structure implementing the cancer detection and subtyping module 630 described in FIG. 6, according to some embodiments. In some embodiments, the cancer detection and subtyping module 630 and/or one or more of its submodules 631-634 may be implemented at least partially via an artificial neural network structure shown in FIG. 7B. The neural network comprises a computing system that is built on a collection of connected units or nodes, referred to as neurons (e.g., 744, 745, 746). Neurons are often connected by edges, and an adjustable weight (e.g., 751, 752) is often associated with the edge. The neurons are often aggregated into layers such that different layers may perform different transformations on the respective input and output transformed input data onto the next layer.

For example, the neural network architecture may comprise an input layer 741, one or more hidden layers 742 and an output layer 743. Each layer may comprise a plurality of neurons, and neurons between layers are interconnected according to a specific topology of the neural network topology. The input layer 741 receives the input data (e.g., 640 in FIG. 6), such as oncRNA training data. The number of nodes (neurons) in the input layer 741 may be determined by the dimensionality of the input data (e.g., the length of a vector of oncRNA count data). Each node in the input layer represents a feature or attribute of the input.

The hidden layers 742 are intermediate layers between the input and output layers of a neural network. It is noted that two hidden layers 742 are shown in FIG. 7B for illustrative purpose only, and any number of hidden layers may be utilized in a neural network structure. Hidden layers 742 may extract and transform the input data through a series of weighted computations and activation functions.

For example, as discussed in FIG. 6 the cancer detection and subtyping module 630 receives an input 740 of oncRNA count data and transforms the input into an output 750 of predicted cancer diagnostics. To perform the transformation, each neuron receives input signals, performs a weighted sum of the inputs according to weights assigned to each connection (e.g., 751, 752), and then applies an activation function (e.g., 761, 762, etc.) associated with the respective neuron to the result. The output of the activation function is passed to the next layer of neurons or serves as the final output of the network. The activation function may be the same or different across different layers. Example activation functions include but not limited to Sigmoid, hyperbolic tangent, Rectified Linear Unit (ReLU), Leaky ReLU, Softmax, and/or the like. In this way, after a number of hidden layers, input data received at the input layer 741 is transformed into rather different values indicative data characteristics corresponding to a task that the neural network structure has been designed to perform.

The output layer 743 is the final layer of the neural network structure. It produces the network's output or prediction based on the computations performed in the preceding layers (e.g., 741, 742). The number of nodes in the output layer depends on the nature of the task being addressed. For example, in a binary classification problem, the output layer may consist of a single node representing the probability of belonging to one class. In a multi-class classification problem, the output layer may have multiple nodes, each representing the probability of belonging to a specific class.

Therefore, the cancer detection and subtyping module 630 and/or one or more of its submodules 631-634 may comprise the transformative neural network structure of layers of neurons, and weights and activation functions describing the non-linear transformation at each neuron. Such a neural network structure is often implemented on one or more hardware processors 710, such as a graphics processing unit (GPU). An example neural network may be a VAE, and/or the like.

In one embodiment, the cancer detection and subtyping module 630 and its submodules 631-634 may be implemented by hardware, software and/or a combination thereof. For example, the cancer detection and subtyping module 630 and its submodules 631-634 may comprise a specific neural network structure implemented and run on various hardware platforms 760, such as but not limited to CPUs (central processing units), GPUs (graphics processing units), FPGAs (field-programmable gate arrays), Application-Specific Integrated Circuits (ASICs), dedicated AI accelerators like TPUs (tensor processing units), and specialized hardware accelerators designed specifically for the neural network computations described herein, and/or the like. Example specific hardware for neural network structures may include, but not limited to Google Edge TPU, Deep Learning Accelerator (DLA), NVIDIA AI-focused GPUs, and/or the like. The hardware 760 used to implement the neural network structure is specifically configured based on factors such as the complexity of the neural network, the scale of the tasks (e.g., training time, input data scale, size of training dataset, etc.), and the desired performance.

In one embodiment, the neural network based cancer detection and subtyping module 630 and one or more of its submodules 631-632 may be trained by iteratively updating the underlying parameters (e.g., weights 751, 752, etc., bias parameters and/or coefficients in the activation functions 761, 762 associated with neurons) of the neural network based on the loss described in relation to FIGS. 2, 3A-3B. For example, during forward propagation, the training data such as oncRNA count data from training samples are fed into the neural network. The data flows through the network's layers 741, 742, with each layer performing computations based on its weights, biases, and activation functions until the output layer 743 produces the network's output 750. In some embodiments, output layer 743 produces an intermediate output on which the network's output 750 is based.

The output generated by the output layer 743 is compared to the expected output (e.g., a “ground-truth” such as the corresponding annotated cancer existence) from the training data, to compute a loss function that measures the discrepancy between the predicted output and the expected output. For example, the loss function may be a triplet margin loss 242, a cross-entropy loss 311, a K-L divergence loss 312, and/or the like. Given the loss, the negative gradient of the loss function is computed with respect to each weight of each layer individually. Such negative gradient is computed one layer at a time, iteratively backward from the last layer 743 to the input layer 741 of the neural network. These gradients quantify the sensitivity of the network's output to changes in the parameters. The chain rule of calculus is applied to efficiently calculate these gradients by propagating the gradients backward from the output layer 743 to the input layer 741.

Parameters of the neural network are updated backwardly from the last layer to the input layer (backpropagating) based on the computed negative gradient using an optimization algorithm to minimize the loss. The backpropagation from the last layer 743 to the input layer 741 may be conducted for a number of training samples in a number of iterative training epochs. In this way, parameters of the neural network may be gradually updated in a direction to result in a lesser or minimized loss, indicating the neural network has been trained to generate a predicted output value closer to the target output value with improved prediction accuracy. Training may continue until a stopping criterion is met, such as reaching a maximum number of epochs or achieving satisfactory performance on the validation data. At this point, the trained network can be used to make predictions on new, unseen data, such as generating a prediction of a cancer diagnose, a tissue of origin, a cancer subtype, etc. in response to an input of oncRNA count.

Neural network parameters may be trained over multiple stages. For example, initial training (e.g., pre-training) may be performed on one set of training data, and then an additional training stage (e.g., fine-tuning) may be performed using a different set of training data. In some embodiments, all or a portion of parameters of one or more neural-network model being used together may be frozen, such that the “frozen” parameters are not updated during that training phase. This may allow, for example, a smaller subset of the parameters to be trained without the computing cost of updating all of the parameters.

Therefore, the training process transforms the neural network into an “updated” trained neural network with updated parameters such as weights, activation functions, and biases. The trained neural network thus improves neural network technology in cancer diagnostics.

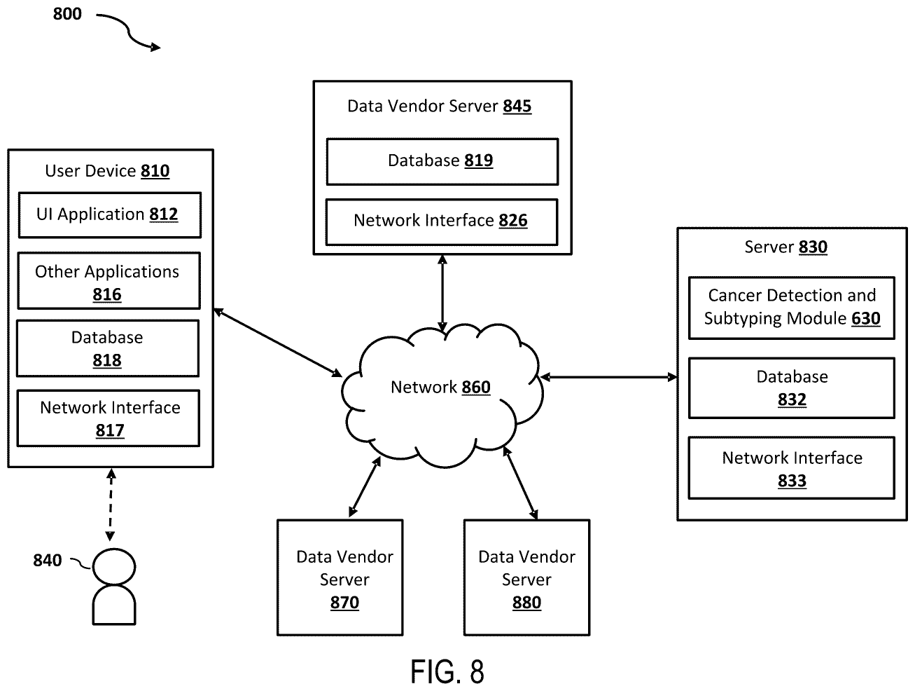

FIG. 8 is a simplified block diagram of a networked system 800 suitable for implementing the cancer detection and subtyping framework described in FIGS. 1A-4 and other embodiments described herein. In one embodiment, system 800 includes the user device 810 which may be operated by user 840, data vendor servers 845, 870 and 880, server 830, and other forms of devices, servers, and/or software components that operate to perform various methodologies in accordance with the described embodiments. Exemplary devices and servers may include device, stand-alone, and enterprise-class servers which may be similar to the computing device 400 described in FIG. 4, operating an OS such as a MICROSOFT® OS, a UNIX® OS, a LINUX® OS, or other suitable device and/or server-based OS. It can be appreciated that the devices and/or servers illustrated in FIG. 8 may be deployed in other ways and that the operations performed, and/or the services provided by such devices and/or servers may be combined or separated for a given embodiment and may be performed by a greater number or fewer number of devices and/or servers. One or more devices and/or servers may be operated and/or maintained by the same or different entities.

The user device 810, data vendor servers 845, 870 and 880, and the server 830 may communicate with each other over a network 860. User device 810 may be utilized by a user 840 (e.g., a driver, a system admin, etc.) to access the various features available for user device 810, which may include processes and/or applications associated with the server 830 to receive an output data anomaly report.

User device 810, data vendor server 845, and the server 830 may each include one or more processors, memories, and other appropriate components for executing instructions such as program code and/or data stored on one or more computer readable mediums to implement the various applications, data, and steps described herein. For example, such instructions may be stored in one or more computer readable media such as memories or data storage devices internal and/or external to various components of system 800, and/or accessible over network 860.

User device 810 may be implemented as a communication device that may utilize appropriate hardware and software configured for wired and/or wireless communication with data vendor server 845 and/or the server 830. For example, in one embodiment, user device 810 may be implemented as an autonomous driving vehicle, a personal computer (PC), a smart phone, laptop/tablet computer, wristwatch with appropriate computer hardware resources, eyeglasses with appropriate computer hardware (e.g., GOOGLE GLASS®), other type of wearable computing device, implantable communication devices, and/or other types of computing devices capable of transmitting and/or receiving data, such as an IPAD® from APPLE®. Although only one communication device is shown, a plurality of communication devices may function similarly.

User device 810 of FIG. 8 contains a user interface (UI) application 812, and/or other applications 816, which may correspond to executable processes, procedures, and/or applications with associated hardware. For example, the user device 810 may receive a message in the form of a medical report, including the predicted presence of cancer, tissue of origin, and subtype, from the server 830 and display the message via the UI application 812. In other embodiments, user device 810 may include additional or different modules having specialized hardware and/or software as required.

In various embodiments, user device 810 includes other applications 816 as may be desired in particular embodiments to provide features to user device 810. For example, other applications 816 may include security applications for implementing client-side security features, programmatic client applications for interfacing with appropriate application programming interfaces (APIs) over network 860, or other types of applications. Other applications 816 may also include communication applications, such as email, texting, voice, social networking, and IM applications that allow a user to send and receive emails, calls, texts, and other notifications through network 860. For example, the other application 816 may be an email or instant messaging application that receives a prediction result message from the server 830. Other applications 816 may include device interfaces and other display modules that may receive input and/or output information. For example, other applications 816 may contain software programs for asset management, executable by a processor, including a graphical user interface (GUI) configured to provide an interface to the user 840 to view the medical report of diagnostic prediction results. The user 840 may be a patient, a medical practitioner, an agent who processes medical results, or the like.

User device 810 may further include database 818 stored in a transitory and/or non-transitory memory of user device 810, which may store various applications and data and be utilized during execution of various modules of user device 810. Database 818 may store user profile relating to the user 840, predictions previously viewed or saved by the user 840, historical data received from the server 830, and/or other types of user-related information. In some embodiments, database 818 may be local to user device 810. However, in other embodiments, database 818 may be external to user device 810 and accessible by user device 810, including cloud storage systems and/or databases that are accessible over network 860.

User device 810 includes at least one network interface component 817 adapted to communicate with data vendor server 845 and/or the server 830. In various embodiments, network interface component 817 may include a DSL (e.g., Digital Subscriber Line) modem, a PSTN (Public Switched Telephone Network) modem, an Ethernet device, a broadband device, a satellite device and/or various other types of wired or wireless network communication devices including microwave, radio frequency, infrared, Bluetooth, and near field communication devices.

Data vendor server 845 may correspond to a server that hosts database 819 to provide training datasets including an oncRNA count dataset to the server 830. The database 819 may be implemented by one or more relational databases, distributed databases, cloud databases, and/or the like.

The data vendor server 845 includes at least one network interface component 826 adapted to communicate with user device 810 and/or the server 830. In various embodiments, network interface component 826 may include a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency, infrared, Bluetooth, and near field communication devices. For example, in one implementation, the data vendor server 845 may send asset information from the database 819, via the network interface 826, to the server 830.

The server 830 may be housed with the cancer detection and subtyping module 630 and its submodules described in FIG. 6. In some implementations, cancer detection and subtyping module 430 may receive data from database 819 at the data vendor server 845 via the network 860 to generate a detection prediction. The generated results may also be sent to the user device 810 for review by the user 840 via the network 860.

In one embodiment, the cancer detection and subtyping module 430 may receive training datasets from multiple vendors 845, 870 and 880. The cancer detection and subtyping module 430 may aggregate multiple datasets from different vendors into a large training dataset for training. As described in relation to FIGS. 1A-1B, the cancer detection and subtyping module 430 uses variational Bayes inference and semi-supervised training to adjust for batch effects or other sources of variation, in this instance resulting from the use of different training data from the three vendors 845, 870 and 880, learn a low dimensional distribution explaining biological variability of the data and classify cancer state, tissue of origin, and cancer subtype.

The database 832 may be stored in a transitory and/or non-transitory memory of the server 830. In one implementation, the database 832 may store data obtained from the data vendor server 845. In one implementation, the database 832 may store parameters of the cancer detection and subtyping module 430. In one implementation, the database 832 may store previously generated prediction results, and the corresponding input feature vectors. In another implementation, the database 832 stores at least two of the foregoing, optionally in combination with at least one additional type of information that may be of value in the present method.

In some embodiments, database 832 may be local to the server 830. However, in other embodiments, database 832 may be external to the server 830 and accessible by the server 830, including cloud storage systems and/or databases that are accessible over network 860.

The server 830 includes at least one network interface component 833 adapted to communicate with user device 810 and/or data vendor servers 845, 870 or 880 over network 860. In various embodiments, network interface component 833 may comprise a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency (RF), and infrared (IR) communication devices.

Network 860 may be a single network or a combination of multiple networks. For example, in various embodiments, network 860 may include the Internet or one or more intranets, landline networks, wireless networks, and/or other appropriate types of networks. Thus, network 860 may correspond to small scale communication networks, such as a private or local area network, or a larger scale network, such as a wide area network or the Internet, accessible by the various components of system 800.

### Example Work Flow

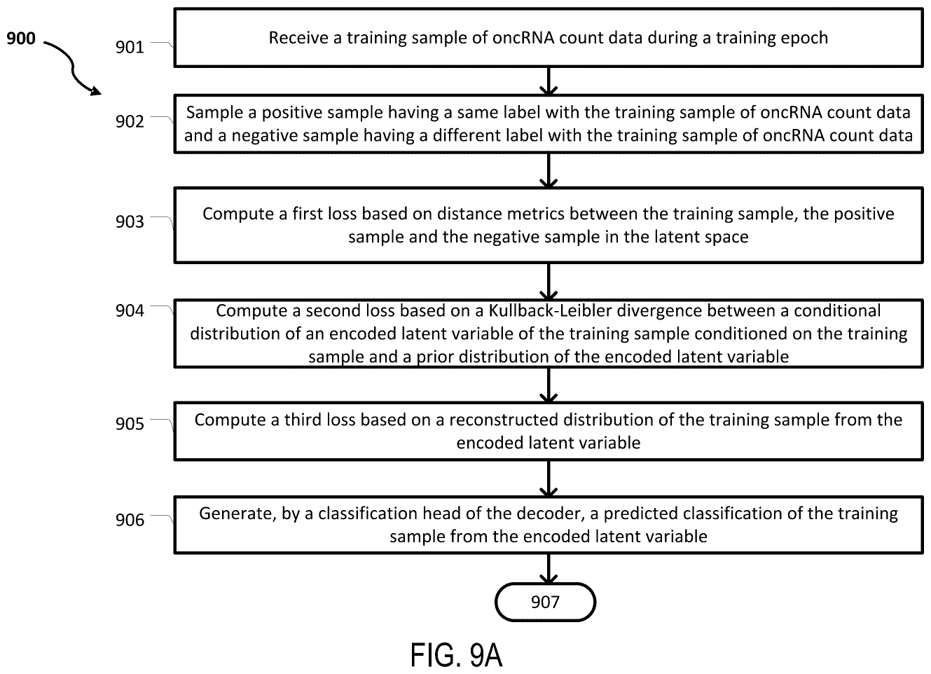

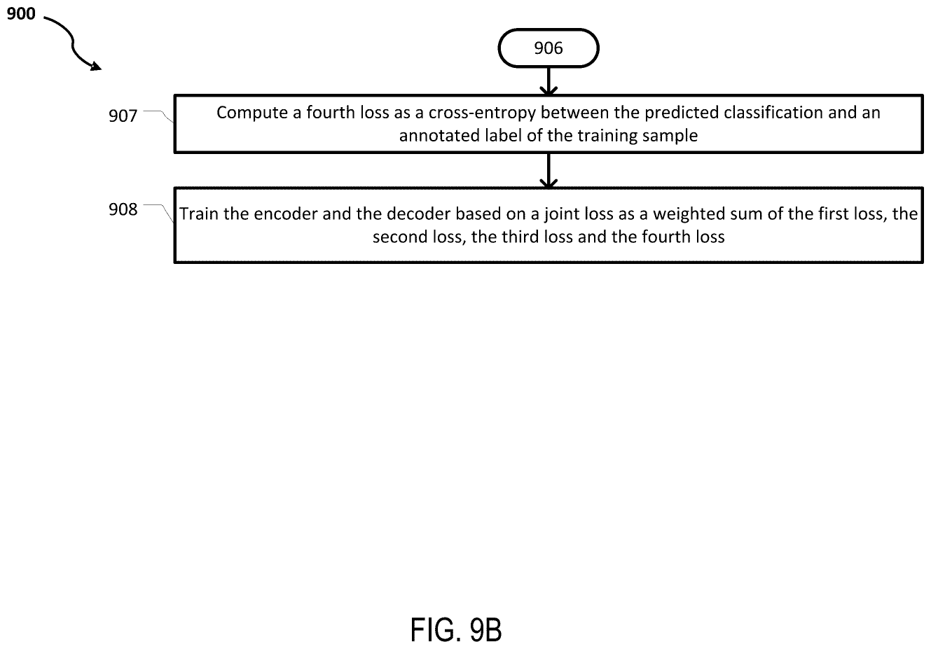

FIGS. 9A-9B provide an example logic flow diagram illustrating a method of training a neural network based model for generating a cancer diagnostic prediction based on the framework shown in FIGS. 1-5, according to some embodiments described herein. One or more of the processes of method 900 may be implemented, at least in part, in the form of executable code stored on non-transitory, tangible, machine-readable media that when run by one or more processors may cause the one or more processors to perform one or more of the processes. In some embodiments, method 900 corresponds to the operation of the cancer detection and subtyping module 630 (e.g., FIGS. 6 and 8) that is trained to generate cancer diagnostic predictions.

As illustrated, the method 900 includes a number of enumerated steps, but aspects of the method 900 may include additional steps before, after, and in between the enumerated steps. In some aspects, one or more of the enumerated steps may be omitted or performed in a different order.

At step 901, a training sample of oncRNA count data (e.g., 201 in FIG. 2) may be received, via a communication interface, during a training epoch.

At step 902, a positive sample having a same label with the training sample of oncRNA count data and a negative sample having a different label with the training sample of oncRNA count data may be sampled from a training batch of samples, e.g., as described in relation to FIG. 3A.

At step 903, a first loss may be computed based on distance metrics between the training sample, the positive sample and the negative sample in the latent space, e.g., such as the triplet margin loss 242 in FIG. 3A.

At step 904, a second loss may be computed based on a Kullback-Leibler divergence between a conditional distribution of an encoded latent variable of the training sample conditioned on the training sample and a prior distribution of the encoded latent variable, e.g., such as the KL-divergence loss 312 in FIG. 3B.

At step 905, a third loss may be computed based on a reconstructed distribution of the training sample from the encoded latent variable (e.g., 231 in FIG. 2). For example, the decoder may generate a reconstructed distribution of the training sample from the encoded latent variable.

At step 906, a classification head (e.g., 310 in FIG. 3B) of the decoder may generate a predicted classification of the training sample from the encoded latent variable.

At step 907, a fourth loss may be computed as a cross-entropy between the predicted classification and an annotated label of the training sample, e.g., cross-entropy loss 311 in FIG. 3B.

At step 908, the encoder and the decoder may be trained jointly based on a joint loss as a weighted sum of the first loss, the second loss, the third loss and the fourth loss. Alternatively, the encoder may be trained based at least in part on the first loss at a first training stage (e.g., as shown in FIG. 3A) and the encoder and the decoder based at least in part on the second loss or the fourth loss at a second training stage after the first training stage (e.g., as shown in FIG. 3B).

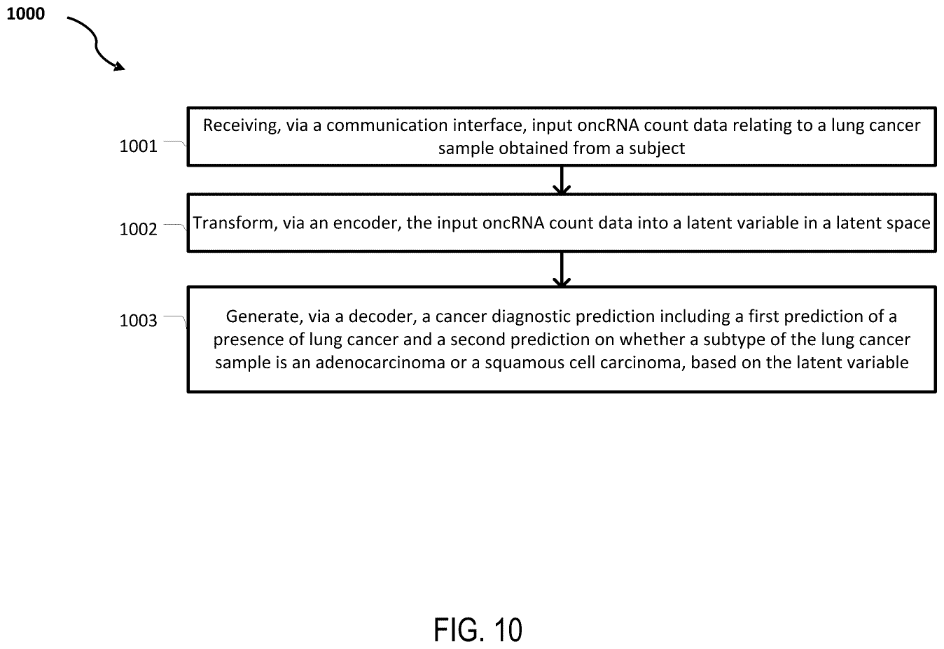

FIG. 10 provides an example logic flow diagram illustrating a method of subtyping a lung cancer sample via a neural network based model based on the framework shown in FIGS. 1-5, according to some embodiments described herein. One or more of the processes of method 900 may be implemented, at least in part, in the form of executable code stored on non-transitory, tangible, machine-readable media that when run by one or more processors may cause the one or more processors to perform one or more of the processes. In some embodiments, method 1000 corresponds to the operation of the cancer detection and subtyping module 630 (e.g., FIGS. 6 and 8) that is trained to generate cancer diagnostic predictions.

As illustrated, the method 1000 includes a number of enumerated steps, but aspects of the method 900 may include additional steps before, after, and in between the enumerated steps. In some aspects, one or more of the enumerated steps may be omitted or performed in a different order.

At step 1001, input oncRNA count data relating to a lung cancer sample obtained from a subject may be received, via a communication interface.

At step 1002, an encoder (e.g., encoder 210 in FIG. 2) may transform the input oncRNA count data into a latent variable (e.g., 231 in FIG. 2) in a latent space (e.g., 230 in FIG. 2).

At step 1003, a decoder (e.g., 220 in FIG. 2) may generate a cancer diagnostic prediction including a first prediction of a presence of lung cancer (e.g., 411 in FIG. 4) and a second prediction (e.g., 413 in FIG. 4) on whether a subtype of the lung cancer sample is an adenocarcinoma or a squamous cell carcinoma, based on the latent variable.

### EXAMPLE

In this example, embodiments described herein were applied for analyzing oncRNAs extracted from serum to investigate their clinical utility for early detection and subtyping (squamous cell carcinoma and adenocarcinoma) of Non-Small Cell Lung Cancer (NSCLC). 887 serum samples were collected from Indivumed (Hamburg, Germany; 222 control samples from individuals with benign disease of the lung, breast, or colon, and 320 cases from individuals with NSCLC) and MT Group (Los Angeles, CA; 345 control samples from individuals without known history of cancer). These samples were collected retrospectively as part of four independent in-house studies. RNA isolated from 0.5 mL of serum from each individual was used to generate and sequence smRNA libraries at an average depth of 18.5±6.5 million 50-bp single-end reads are used.

The Cancer Genome Atlas (TCGA) smRNA-seq database and an in-house reference serum cohort of non-cancer donors (for filtration of bona fide smRNAs) were used to identify 255,953 distinct NSCLC-specific oncRNA species. After processing serum samples for the present study, 185,905 (72.6%) oncRNAs were detected in at least one sample.

To model samples from multiple suppliers and studies, the customized semi-supervised, generative AI model described herein was used for statistical inference, batch correction, and prediction of cancer's presence as well as its subtype. For comparison, a standard linear model with elastic net regularization was used as baseline.

Clinical cohorts for training datasets used for the training process described in FIGS. 3A-3B were as follows:

| Demographics | | Non-cancer | | Lung | |
|---|---|---|---|---|---|
| Sample | | Controls | | Cancer | |
| size | N | 567 | | 320 | |
| Age | Mean (SD) | 61.7 | (11.7) | 65.2 | (9.81) |
| | ≥65 years, N (%) | 269 | (47.4%) | 174 | (54.4%) |
| Sex | Female (%) | 255 | (45%) | 120 | (37.5%) |
| Smoking | Never-Smoker, N (%) | 236 | (41.6%) | 17 | (5.31%) |
| | Ever-Smoker, N (%) | 98 | (17.3%) | 217 | (67.8%) |
| BMI | BMI <30, N (%) | 439 | (77.4%) | 261 | (81.6%) |
| | BMI ≥30, N (%) | 127 | (22.4%) | 59 | (18.4%) |
| Ethnicity | White/non-hispanic, N | 324 | (57.1%) | 315 | (98.4%) |
| | (%) | | | | |
| | Black/African American, | 2 | (0.353%) | 0 | (0%) |
| | N (%) | | | | |
| | Asian/Hispanic/Others, | 241 | (42.5%) | 5 | (1.56%) |
| | N (%) | | | | |

Lung cancer cohort

| Sample size | N | 320 | |
|---|---|---|---|
| Overall clinical stage | Stage I | 138 | (43.1%) |
| | Stage II | 82 | (25.6%) |
| | Stage III | 77 | (24.1%) |
| | Stage IV | 23 | (7.19%) |
| Lung cancer subgroup | Adenocarcinoma | 184 | (57.5%) |
| | Squamous cell carcinoma | 136 | (42.5%) |

Workflow for smRNA-seq of RNA isolated from serum: (i) Zymo Research Quick-cfRNA serum & plasma kit (cat no R105) was used to isolate RNA from 1 mL of serum per manufacturer's protocol; (ii) Takara SMARTer smRNA-Seq kit from Illumina (cat no 635031) was used to generate libraries from the RNA isolated in step (i); and (iii) the libraries generated in step (ii) were sequenced on an Illumina NextSeq2000 instrument.

Using 10-fold cross-validation, the AI-based tool described herein and linear models, the AUCs were 0.98 (95% CI: 0.97-0.99) and 0.85 (0.82-0.87), respectively. More importantly, stage I sensitivity was 0.88 (0.82-0.93) for the AI model vs 0.36 (0.28-0.45) for the linear model at 95% specificity. Sensitivity for later stages (II, III, and IV) was 0.93 (0.88-0.96) and 0.42 (0.35-0.49) for the AI and linear models, respectively. For detecting tumors smaller than 2 cm (T1a-b), the AI model achieved a sensitivity of 0.85 (0.73-0.94) at 95% specificity, while the linear model had a sensitivity of 0.35 (0.22-0.49).

The AI-based tool was also trained to distinguish squamous cell carcinoma from adenocarcinoma for late stage (III/IV) NSCLC using small RNA content of the serum. It achieved sensitivity of 0.67 (0.53-0.8) at 70% specificity, while the linear model had sensitivity of 0.46 (0.32-0.6) at 70% specificity.

Therefore, these results demonstrate that oncRNA profiling and the AI-based tool described herein may be applied for accurate, sensitive, and early detection of NSCLC through sequencing of a routine blood draw sample. Additionally, the AI model can subtype NSCLC directly from the serum, establishing the role of oncRNAs as non-invasive biomarkers predictive of patient outcomes.

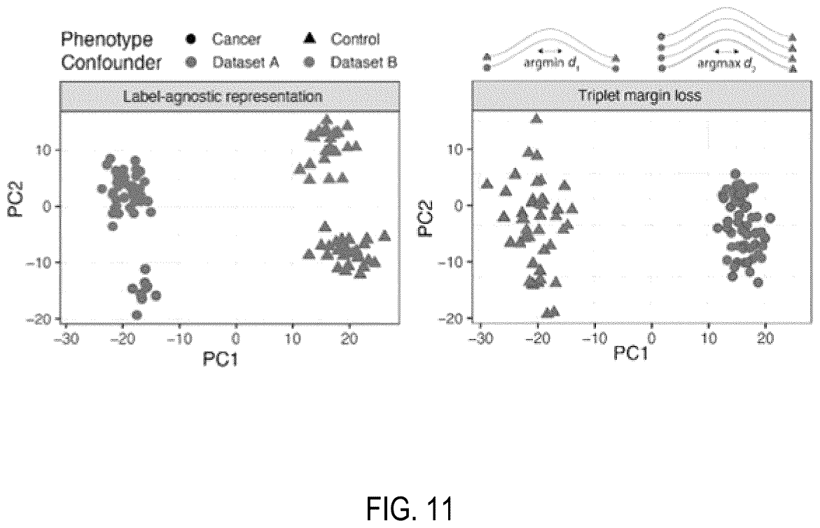

FIG. 11 provides an example illustration of triplet margin loss application on simulated data. Left panel shows a label-agnostic embedding, and the right panel shows an embedding with a triplet margin loss constraint to minimize technical variations while preserving biological differences. For each sample, positive anchors (same phenotype, different dataset) and negative anchors (different phenotype, any dataset) are sampled to minimize or maximize the embedding distance, respectively.

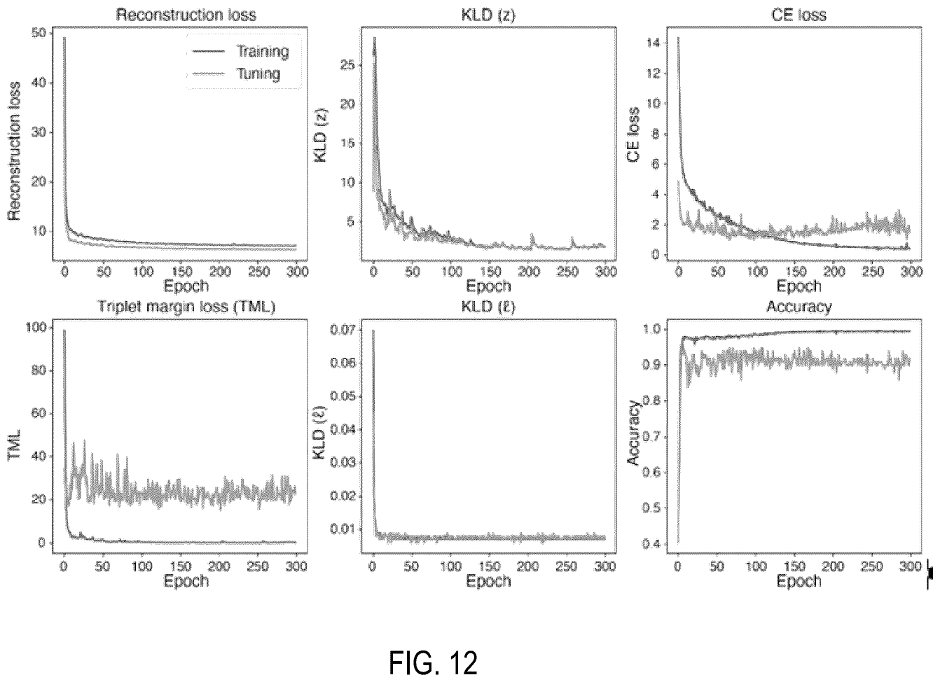

FIG. 12 provides example loss convergence plots showing the convergence of the reconstruction loss, K-L divergence loss based on latent variable z 231, cross-entropy loss, triplet margin loss, K-L divergence loss based on latent variable l 232 as described in relation to FIG. 2, as well classification accuracy during training.

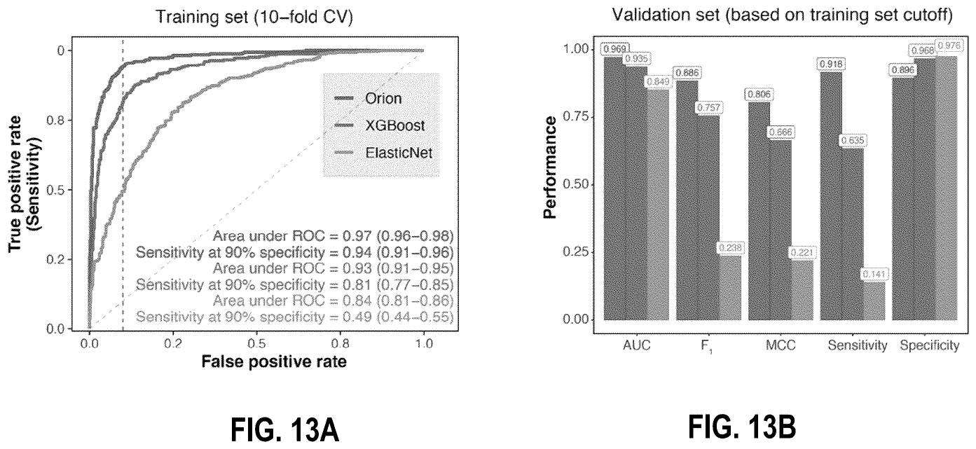

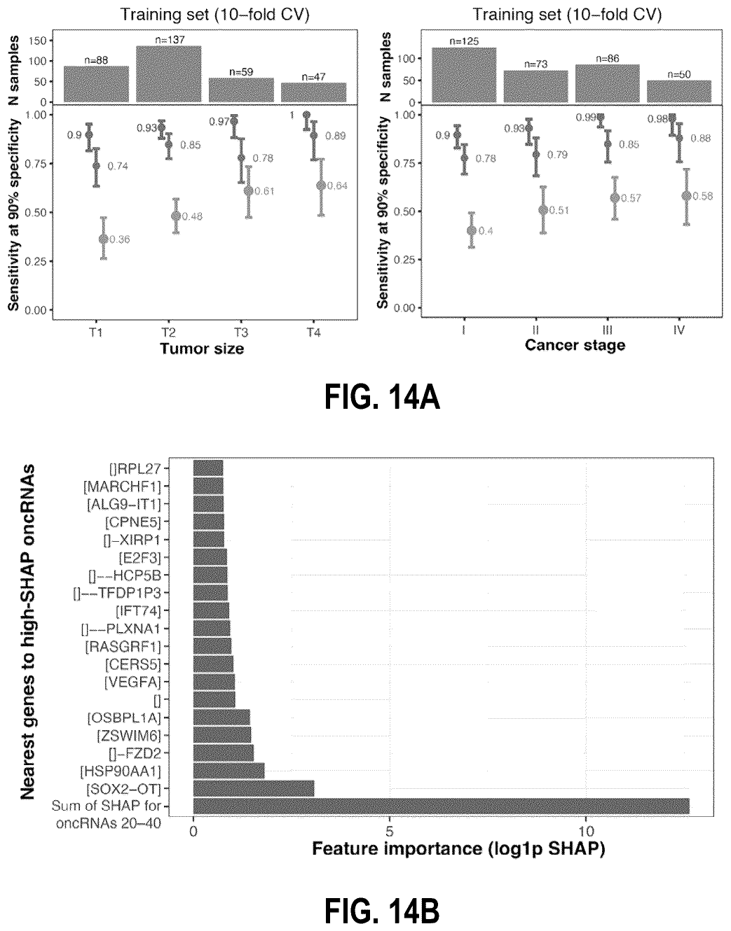

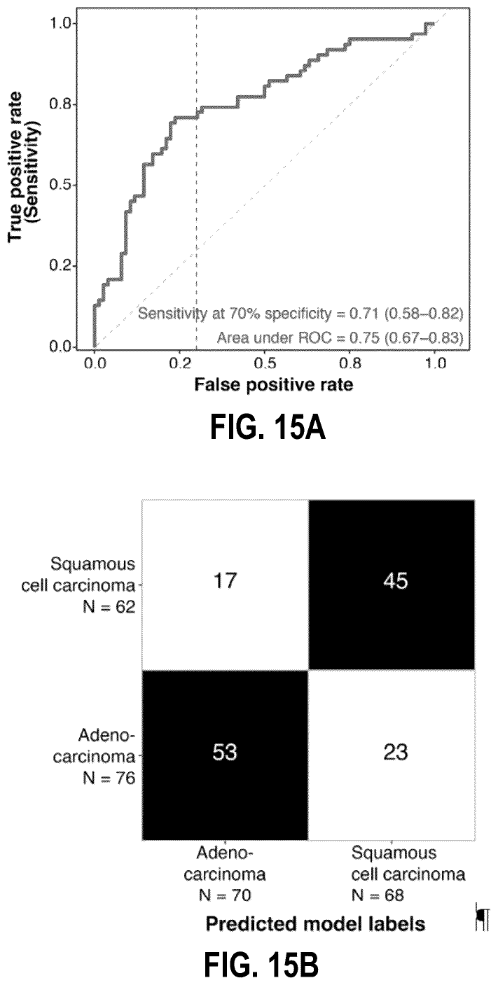

FIG. 13A-14B illustrate example performance of the cancer detection and subtyping neural network model described in FIGS. 1-10. To evaluate the performance of cancer detection and subtyping neural network model, dataset may be divided into a held-out 20% and a remaining 80%. For 80% of the data, the cancer detection and subtyping neural network model may be trained in a non-overlapping 10-fold cross validation setup. During each fold, a subset of TCGA-derived oncRNAs that within the training set, were enriched among the cancer samples compared to control samples of each data source supplier, resulting in an average of 6,376±60 (S.D) oncRNAs per fold. Five cancer detection and subtyping neural network models with different random seeds are trained on each fold and averaged the scores on the test set.

As shown in FIG. 13A, the cancer detection and subtyping neural network model (referred to as “Orion” in FIG. 13A) achieves area under Receiver-operating characteristic curve (ROC) of 0.97 (95% CI 0.96-0.98) and overall sensitivity of 92% (88%-95%) at 90% specificity. In an identical setup with the same set of oncRNAs for each training fold, the commonly used ElasticNet model had an area under ROC of 0.84 (0.81-0.86) and overall sensitivity of 49% (44%-55%). Other methods such as XGBoost, k-nearest neighbors classifier and support vector machine classifier also performed worse than the cancer detection and subtyping neural network model. More importantly, stage I sensitivity (n=88) was 0.9 (0.83-0.94) for cancer detection and subtyping neural network model versus 0.4 (0.31-0.49) for the ElasticNet model at 90% specificity.

FIG. 13B shows performance measures of binary classification in the held-out validation set. All threshold-dependent metrics (all except area under ROC) are computed based on the cutoff resulting in 90% specificity in the 10-fold cross validated training dataset. Bar height shows the point estimate of area under ROC, F1 score, Matthew's correlation coefficient (MCC), sensitivity, and specificity. To assess the generalizability of the cancer detection and subtyping neural network model, a cutoff corresponding to 90% specificity is chosen in the 10-fold cross validation, and measured various classification metrics on the held-out validation set. The cancer detection and subtyping neural network model demonstrated a strong agreement in performance for the held-out validation set, while XGBoost and ElasticNet performances were on the lower bound of its 10-fold CV measurements. In a bootstrap analysis, AUC of the cancer detection and subtyping neural network model was significantly higher than ElasticNet as well (Δ_{AUC}=0.13 (95% CI: 0.11-0.16). While AUC of Orion and XGBoost were relatively similar (Δ_{AUC}=0.04 (0.03-0.05)), F₁ score, sensitivity of the cancer detection and subtyping neural network model at 90% specificity, and generalizability to validation set were also better for Orion compared to XGBoost (Δ_{F}1=0.07 (0.04-0.1), Δ_{sensitivity}=0.12 (0.08-0.16).

As shown in FIG. 14A, sensitivity for later stages (II, III, and IV with n=243) was 0.97 (0.93-0.99) and 0.55 (0.48-0.62) for the cancer detection and subtyping neural network model and the ElasticNet model, respectively. Detecting tumors smaller than 2 cm (T1a-b, n=52), the cancer detection and subtyping neural network model achieves a sensitivity of 0.87 (0.74-0.94) at 90% specificity, while the ElasticNet model had a sensitivity of 0.31 (0.19-0.59) at 90% specificity.

As a measure of successful batch effect removal, the model scores for control samples are expected to be similar, and therefore, not distinguish the sample suppliers. The cancer detection and subtyping neural network model had an area under ROC of 0.53 (0.47-0.58), suggesting it successfully removed the impact of suppliers, while XGBoost and ElasticNet had higher area under ROCs of 0.62 (0.57-0.670) and 0.59 (0.54 0.64), respectively.

Given that the control samples in the cohort had an over-representation of individuals without smoking history compared to the cancer samples (54% vs. 10%), the impact of smoking status of samples are examined on model scores. It is found that among control samples, the cancer detection and subtyping neural network model validation set score had an area under ROC of 0.6 (0.5-0.7) with respect to presence of smoking history, further confirming little variation of the model score for individuals with or without a history of smoking.

To identify the most important oncRNAs for the cancer detection and subtyping neural network model, Shapley Additive exPlanations (SHAP) (Lundberg et al., a unified approach to interpreting model predictions, Advances in Neural Information Processing Systems, volume 30. Curran Associates, Inc., 2017) average values among model folds. As shown in FIG. 14B, among the high-SHAP oncRNAs for the model, it is observed that overlap or vicinity of oncRNAs to some of the genes with significance in lung cancer etiology and prognosis. These included SOX2-OT (Dodangeh et al., Long non-coding RNA SOX2-OT enhances cancer biological traits via sponging to tumor suppressor mir-122-3p and mir-194-5p in non-small cell lung carcinoma, Scientific Reports, 13 (1): 12371, 2023), HSP90AA1, (Niu et al., Targeting hsp90 inhibits proliferation and induces apoptosis through akt 1/erk pathway in lung cancer, Frontiers in Pharmacology, 12:724192, 2022; Bhattacharyya et al., CDK1 and HSP90AA1 appear as the novel regulatory genes in non-small cell lung cancer: a bioinformatics approach, Journal of Personalized Medicine, 12 (3): 393, 2022), and FZD2 (Tuluhong et al., Fzd2 promotes tgf-β-induced epithelial-to-mesenchymal transition in breast cancer via activating notch signaling pathway, Cancer Cell International, 21:1-13, 2021).

FIGS. 15A-15B provide example performance charts illustrating tumor subtyping performance from circulating oncRNAs. In addition to the early detection of cancer signals in patients with NSCLC, understanding the tumor histology has major implications in therapy selection and resistance mechanisms. Squamous cell carcinoma transformation of lung adenocarcinoma has been reported to take place after target therapy resistance. Squamous cell carcinoma transformation has been reported to be one of the mechanisms of acquired resistance to epidermal growth factor receptor (EGFR). Traditional methods of stratifying patients to evaluate for squamous cell carcinoma transformation involve repeat biopsies of a lung cancer patient which can lead to severe side effects such as pneumothorax, hemorrhage, and air embolism.

Given the tissue-specific landscape of chromatin accessibility in different cancers, oncRNA expression patterns are unique to cancer types and subtypes, allowing the model to detect tissue of origin among different cancer types non-invasively from blood. It is hypothesized that biological differences of lung adenocarcinoma and squamous cell carcinoma would also be reflected in serum oncRNA content, allowing the model to distinguish these major subtypes of NSCLC. While tumor tissues are vastly different from normal tissue, the differences in subtypes of a given tumor are far less substantial. In NSCLC, for example, the agreement of pathologists for different subtypes is approximated to be 0.81. As a result, tumor histology subtype prediction is more difficult than cancer detection.

To evaluate the hypothesis, the potential of distinguishing two major NSCLC subtypes are investigated, adenocarcinoma and squamous cell carcinoma, using oncRNAs in blood. For this analysis, 20-fold cross validation to adjust for the reduction in number of samples given that this is a NSCLC-specific task.

FIG. 15A shows a ROC plot of Orion for distinguishing squamous cell carcinoma from adenocarcinoma among stage III/IV NSCLC samples. FIG. 15B shows a confusion matrix of Orion's subtype prediction at 70% specificity cutoff. For later stage tumors (stages III/IV), the cancer subtyping model achieved area under ROC of 0.75 (95% CI: 0.67-0.83) and a sensitivity of 0.71 (95% CI: 0.56-0.84) at 70% specificity in distinguishing squamous cell carcinoma from adenocarcinoma samples in serum samples, as shown in FIGS. 15A-15B.

This description and the accompanying drawings that illustrate inventive aspects, embodiments, implementations, or applications should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure the embodiments of this disclosure. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

What is claimed is:

1. A method of generating a cancer diagnostic prediction via a neural network based model implemented on one or more hardware processors, the method comprising:
receiving, via a communication interface, a plurality of samples of orphan non-coding ribonucleic acid (oncRNA) count data;
transforming, via an encoder, a sample of oncRNA count data into a latent variable in a latent space; and
generating, via a decoder, a cancer diagnostic prediction based on the latent variable.

2. The method of claim 1, wherein the plurality of samples of oncRNA count data are received from one or more data sources.

3. The method of claim 1, wherein the encoder comprises one or more of:
a first variational autoencoder (VAE) that encodes a first sample relating to ribonucleic acid (RNA) subtypes used for classification; and
a second VAE that encodes a second sample relating to endogenous highly expressed RNA biotypes used for library estimation.

4. The method of claim 1, wherein the cancer diagnostic prediction comprises any of:
a presence of cancer;
a tissue of origin; and
a cancer subtype.

5. The method of claim 1, further comprising:
receiving a training sample of oncRNA count data during a training epoch;
sampling a positive sample having a same label with the training sample of oncRNA count data;
sampling a negative sample having a different label with the training sample of oncRNA count data; and
computing a first loss based on distance metrics between the training sample, the positive sample and the negative sample in the latent space.

6. The method of claim 5, further comprising:
computing a second loss based on a Kullback-Leibler divergence between a conditional distribution of an encoded latent variable of the training sample conditioned on the training sample and a prior distribution of the encoded latent variable.

7. The method of claim 6, further comprising:
generating, by the decoder, a reconstructed distribution of the training sample from the encoded latent variable; and
computing a third loss based on the reconstructed distribution.

8. The method of claim 7, further comprising:
generating, by a classification head of the decoder, a predicted classification of the training sample from the encoded latent variable; and
computing a fourth loss as a cross-entropy between the predicted classification and an annotated label of the training sample.

9. The method of claim 8, further comprising:
training the encoder and the decoder based on a joint loss as a weighted sum of the first
loss, the second loss, the third loss and the fourth loss.

10. The method of claim 8, further comprising:
training the encoder based at least in part on the first loss at a first training stage; and
training the encoder and the decoder based at least in part on the second loss or the fourth loss at a second training stage after the first training stage.

11. The method of claim 1, wherein the sample of oncRNA count data relates to a lung cancer sample, and the cancer diagnostic prediction includes a prediction of a detection of a presence of lung cancer, and a prediction of a lung cancer subtype of adenocarcinoma and squamous cell carcinoma.

12. A system of generating a cancer diagnostic prediction via a neural network based model, the system comprising:
a communication interface that receives a plurality of samples of orphan non-coding ribonucleic acid (oncRNA) count data;
a memory storing the neural network based model and a plurality of processor-executable instructions; and
one or more processors that execute the plurality of processor-executable instructions to perform operations comprising:
transforming, via an encoder, a sample of oncRNA count data into a latent variable in a latent space; and
generating, via a decoder, a cancer diagnostic prediction based on the latent variable.

13. The system of claim 12, wherein the plurality of samples of oncRNA count data are received from one or more data sources.

14. The system of claim 12, wherein the encoder comprises one or more of:
a first variational autoencoder (VAE) that encodes a first sample relating to ribonucleic acid (RNA) subtypes used for classification; and
a second VAE that encodes a second sample relating to endogenous highly expressed RNA biotypes used for library estimation.

15. The system of claim 12, wherein the cancer diagnostic prediction comprises any of:
a presence of cancer;
a tissue of origin; and
a cancer subtype.

16. The system of claim 12, wherein the operations further comprise:
receiving a training sample of oncRNA count data during a training epoch;
sampling a positive sample having a same label with the training sample of oncRNA count data;
sampling a negative sample having a different label with the training sample of oncRNA count data; and
computing a first loss based on distance metrics between the training sample, the positive sample and the negative sample in the latent space;
computing a second loss based on a Kullback-Leibler divergence between a conditional distribution of an encoded latent variable of the training sample conditioned on the training sample and a prior distribution of the encoded latent variable;
generating, by the decoder, a reconstructed distribution of the training sample from the encoded latent variable;
computing a third loss based on the reconstructed distribution;
generating, by a classification head of the decoder, a predicted classification of the training sample from the encoded latent variable; and
computing a fourth loss as a cross-entropy between the predicted classification and an annotated label of the training sample.

17. The system of claim 16, wherein the operations further comprise:
training the encoder and the decoder based on a joint loss as a weighted sum of the first loss, the second loss, the third loss and the fourth loss.

18. The system of claim 12, wherein the sample of oncRNA count data relates to a lung cancer sample, and the cancer diagnostic prediction includes a prediction of a detection of a presence of lung cancer, and a prediction of a lung cancer subtype of adenocarcinoma and squamous cell carcinoma.

19. A method of subtyping a lung cancer sample via a neural network based model implemented on one or more hardware processors, the method comprising:
receiving, via a communication interface, input oncRNA count data relating to a lung cancer sample obtained from a subject;
transforming, via an encoder, the input oncRNA count data into a latent variable in a latent space; and
generating, via a decoder, a cancer diagnostic prediction including a first prediction of a presence of lung cancer and a second prediction on whether a subtype of the lung cancer sample is an adenocarcinoma or a squamous cell carcinoma, based on the latent variable.

20. A method of cancer diagnostic and treatment prediction via a neural network based model implemented on one or more hardware processors, the method comprising:
generating, by the neural network based model that transforms oncRNA count data into a latent variable, a cancer diagnostic prediction based on the latent variable; and
generating a recommended treatment when the cancer diagnostic prediction indicates a presence of cancer.
